SEQUENCE LISTING (18 sequences)

SEQ ID NO: 1 - protein, 1108 aa, Leuconostoc citreum
  REGION 1..1108: note = MISC_FEATURE - GBD-CD2deltaN123
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDAWFQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 2 - protein, 1877 aa, Leuconostoc citreum
  REGION 1..1877: note = MISC_FEATURE - BRS-A
MRQKETITRKKLYKSGKSWVAAATAFAVMGVSAVTTVSADTQTPVGTTQSQQDLTGQRGQDKPTTKEVIDKKEPVPQVSAQNAGDLSADAKTTKADDKQDTQPTNAQLPDQGNKQTNSNSDKGVKESTTAPVKTTDVPSKSVTPETNTSINGGQYVEKDGQFVYIDQSGKQVSGLQNIEGHTQYFDPKTGYQTKGELKNIDDNAYYFDKNSGNGRTFTKISNGSYSEKDGMWQYVDSHDKQPVKGLYDVEGNLQYFDLSTGNQAKHQIRSVDGVTYYFDADSGNATAFKAVTNGRYAEQTTKDKDGNETSYWAYLDNQGNAIKGLNDVNGEIQYFDEHTGEQLKGHTATLDGTTYYFEGNKGNLVSVVNTAPTGQYKINGDNVYYLDNNNEAIKGLYGINGNLNYFDLATGIQLKGQAKNIDGIGYYFDKDTGNGSYQYTLMAPSNKNDYTQHNVVNNLSESNFKNLVDGFLTAETWYRPAQILSHGTDWVASTDKDFRPLITVWWPNKDIQVNYLRLMQNEGVLNQSAVYDLNTDQLLLNEAAQQAQIGIEKKISQTGNTDWLNNVLFTTHDGQPSFIKQQYLWNSDSEYHTGPFQGGYLKYQNSDLTPNVNSKYRNADNSLDFLLANDVDNSNPIVQAEDLNWLYYLLNFGSITTQGKENNSNFDSIRIDAVDFVSNDLIQRTYDYLRAAYGVDKNDKEANAHLSLVEAGLDAGTTTIHQDALIESDIREAMKKSLTNGPGSNISLSNLIQDKEGDKLIADRANNSTENVAIPNYSIIHAHDKDIQDKVGAAITDATGADWTNFTPEQLQKGLSLYYEDQRKIEKKYNQYNIPSAYALLLTNKDTVPRVYYGDMYQDDGQYMQKQSLYFDTITALMEARKQFVAGGQTINVDDNGVLTSVRFGKGAMTANDIGTNETRTQGIGVVIANDPSLKLSKDSKVTLHMGAAHRNQNYRALLLTTDNGIDSYSSSKNAPVIKTDDNGDLVFSNQDINDQLNTKVHGFLNSEVSGYLSAWVPLDATEQQDARTLPSEKSVNDGKVLHSNAALDSNLIYEAFSNFQPMPTNRNEYTNVVIADKADTFKSWGITSFEMAPQYRSSQDKTFLDSTIDNGYAFTDRYDLGFEKPTKYGNDEDLRQAIKQLHSSGMQVMADVVANQIYNLPGKEVASTNRVDWNGNNLSTPFGTQMYVVNTVGGGKYQNKYGGEFLDKLKAAYPDIFRSKNYEYDVKNYGGNGTGSVYYTVDSKTRAELDTDTKIKEWSAKYMNGTNVLGLGMGYVLKDWQTGQYFNVSNQNMKFLLPSDLISNDITVQLGVPVTDKKIIFDPASAYNMYSNLPEDMQVMDYQDDKKSTPSIKPLSSYNNKQVQVTRQYTDSKGVSWNLITFAGGDLQGQKLWVDSRALTMTPFKTMNQISFISYANRNDGLFLNAPYQVKGYQLAGMSNQYKGQQVTIAGVANVSGKDWSLISFNGTQYWIDSQALNTNFTHDMNQKVFVNTTSNLDGLFLNAPYRQPGYKLAGLAKNYNNQTVTVSQQYFDDQGTVWSQVVLGGQTVWVDNHALAQMQVRDTNQQLYVNSNGRNDGLFLNAPYRGQGSQLIGMTADYNGQHVQVTKQGQDAYGAQWRLITLNNQQVWVDSRALSTTIMQAMNDDMYVNSSQRTDGLWLNAPYTMSGAKWAGDTRSANGRYVHISKAYSNEVGNTYYLTNLNGQSTWIDKRAFTATFDQVVALNATIVARQRPDGMFKTAPYGEAGAQFVDYVTNYNQQTVPVTKQHSDAQGNQWYLATVNGTQYWIDQRSFSPVVTKVVDYQAKIVPRTTRDGVFSGAPYGEVNAKLVNMATAYQNQVVHATGEYTNASGITWSQFALSGQEDKLWIDKRALQA

SEQ ID NO: 3 - protein, 1274 aa, Leuconostoc citreum
  REGION 1..1274: note = MISC_FEATURE - BRS-Bdelta1
DTQTPVGTTQSQQDLTGQTGQDKPTTKEVIDKKEPVPQVSAQNVGDLSADAKTPKADDKQDTQPTNAQLPDQGNKQTNSNSDKGVKESTTAPVKTTDVPSKSVAPETNTSINGGQYVEKDGQFVYIDQSGKQVSGLQNIEGHTQYFDPKTGYQTKGELKNIDDNAYYFDKNSGNGRTFTKISNGSYSEKDGMWQYVDSHDKQPVKGLYDVEGNLQYFDLSTGNQAKHQIRSVDGVTYYFDADSGNATAFKAVTNGRYAEQTTKDKDGNETSYWAYLDNQGNAIKGLNDVNGEIQYFDEHTGEQLKGHTATVDGTTYYFEGNKGNLVSVVNTAPTGQYKINGDNVYYLDNNNEAIKGLYGINGNLNYFDLATGIQLKGQAKNIDGIGYYFDQNNGNGEYRYSLTGPVVKDVYSQHNAVNNLSANNFKNLVDGFLTAETWYRPAQILSHGTDWVASTDKDFRPLITVWWPNKDIQVNYLKLMQQIGILDNSVVFDTNNDQLVLNKGAESAQIGIEKKVSETGNTDWLNELLFAPNGNQPSFIKQQYLWNVDSEYPGGWFQGGYLAYQNSDLTPYANTNPDYRTHNGLEFLLANDVDNSNPVVQAEQLNWLYYLMNFGQITANDSNANFDSMRIDAISFVDPQIAKKAYDLLDKMYGLTDNEAVANQHISIVEAPKGETPITVEKQSALVESNWRDRMKQSLSKNATLDKLDPDPAINSLEKLVADDLVNRSQSSDKDSSTIPNYSIVHAHDKDIQDTVIHIMKIVNNNPNISMSDFTMQQLQNGLKAFYEDQHQSVKKYNQYNIPSAYALLLTNKDTVPRVFYGDMYQDYGDDLDGGQYMATKSIYYNAIEQMMKARLKYVAGGQIMAVTKIKNDGINKDGTNKSGEVLTSVRFGKDIMDAQGQGTAESRNQGIGVIVSNSSGLELKNSDSITLHMGIAHKNQAYRALMLTNDKGIVNYDQDNNAPIAWTNDHGDLIFTNQMINGQSDTAVKGYLNPEVAGYLAVWVPVGANDNQDARTVTTNQKNTDGKVLHTNAALDSKLMYEGFSNFQKMPTRGNQYANVVITKNIDLFKSWGITDFELAPQYRSSDGKDITDRFLDSIVQNGYGLSDRYDLGFKTPTKYGTDQDLRKAIERLHQAGMSVMADFVANQIYGLHADKEVVSAQHVNINGDTKLVVDPRYGTQMTVVNSVGGGDYQAKYGGEYLDTISKLYPGLLLDSNGQKIDLSTKIKEWSAKYLNGSNIPQVGMGYVLKDWNNGQYFHILDKEGQYSLPTQL

SEQ ID NO: 4 - protein, 1774 aa, Leuconostoc fallax
  REGION 1..1774: note = MISC_FEATURE - BRS-C
MKQQESITRKKLYKAGKSWVVAATLFAATLFAAMGAAGATTVASADVQKDTVVVTADKNTTDKDKEPIKTAGANVVDKGVAQTTDTNTTDKKTIEVGKSVDMSATDKKVTETVKSVDTSATDKKTTEAVKPVDTNATDKKATEAVKPVDTNATDKKTTEAVKPVDTNTTDKKVTEAIKPVNTNADDKTAEPVKTISATKDTVKTIANKQKGATEEQAVITEGHYEAQGDGFVYITKDGKQLTGLQNINGNTQYFDPATGQQLKGDIKAVAGTVYYFDKNSGNARVYQKVADGTYSENNEHWQYISKVDNKPVEGLYNVQGNLQYFDMSTGNQVKNDIRSVDGVTYYFDKDSGNGSAFNALSAGEYVEKKETDAQGNQNSYWTYSGLDGNPVKGLYDINGSLQYFDEKNGAQLKGGTATVNGVTYYFEQDKGNLISVVNSVESGQYKIDNDNVYYIDNQGNTLKGLYAINGQLNYFDMSTGVQLKGASENANGVGYYFDKDKGNGQYQYSLITSTLANAFSKHNAANDYTQSSFTHTVDGFLTADTWYRPTEILKNGTTWVASTSQDLRPMITVWWPNKNVQLNYLKLMQTEGLLDSGQVYDLNSDQALLNQAAQTVQVNIEKRITKAGNSDWLNDLLYNSHGETPSFVKQQAIWNADSEYHGGWFQGGYLAYRNSDLTPYANSSYRHYTGMEFLLANDVDNSNPIVQAEDLNWLYYLMNFGTETGNDPQANFDSIRIDAISFVDKQVAKKAYELLHDMYGLSASDAVANKHVSIVEASADQTPVTTENHDALIESYWRDTMKNSLSKDASIDSSAGSLSAMINDGNVDRANDSTTESSIFPNYTIVHAHDKDIQDAVSNVMKIVNNDPSISLDGFTMEQLEKGLSAFYADQRSAVKQYNQYNIPSAYAVMLTNKDTVPRTFYGDMYQDDGQYMANKSLYYDAIDTMMKARLKYVSGGQTMSVTKINNANSQKSGEVLTSVRFGKGVMDATDAGSAESRTQGIGVVVSNSSGLQLNDNDKIVLHMGAAHKNQEYRALMLTTNDGIKSFNNDEAPINYTDDNGDLIFDGHNIDGQENTAIRGYLNPQVAGYLAVWVPTGAKDDQDARTQPSNEKSTDGKVLHTNAALDSELIYEGFSNFQPMPTTKDEYTNVMIAKNIDLFKSWGITNFELAPQYRSSDGKNINDRFIDSLVQNGYGLSDRYDLGFETPTKYGTDQDLRTAIKTLHQAGMTVMADYVANQIYGLNTSQEVVDAQRVNSDNNAVEVRYGQHLNVVNSIGGGEYQNLYGGKYLEILNKLYPDLLVDENGNKIDIDTKIKQWSAKYLNGSNVTGLGMGYVLKDWSNGQYFNISNTDGKVMLPEQLVKHMPAVEIGTQTNYTAYISSTIRRDGLYNNMPWGVTATGQDGNEIKWERQGSTSDYNHQKVQVNRQYVDKQGVVWNLINFDDKDLWVDSNALVTVNFTSQKPTKHFVQFGMRQGKYDGFYLSAPYKQTESKWVASTRTHQGQLLEVVGQYTTGSGSRKVTWYLVGLDGKQVWVDSRAVGTNFSHKTNINLLINSATRNDGMYLNAPYGQKGYKRETSSRFYNEKLVTVSQQYYDNKGVIWNLITLNGKKLWVDSRAFATVIDKKVNQSLYINSRNDGMYLNAPYRAQGAKRYASTKTYTGQRVQVTLQRKDTHGVTWYLTKVDSKQLWVDSHAFAPTFTRNVSLNVKVNSSKRNDGIYLNAPYGNKKAKRIASTKAYNGKRVKASKEYKDAKGVTWYLVNLNNKQVWIDKRAF

SEQ ID NO: 5 - protein, 1375 aa, Lactobacillus kunkei
  REGION 1..1375: note = MISC_FEATURE - BRS-D_delta 1
NGQTWKYVSQNKDIQGLYKDNNDQLRYFNEYDGTQAKGDIVNVNNDNYYFDKDSGQGHKIDSYTGGSYSESKVNNQDGWIYKSSDNNDVKGVATVDGNIQYFDQNTGLQLKGGSAQIGGVDYYFDPNKGNLVGKVDQVVNSNDYSDNKLLDSNKNVVKGLVVNNGQLQFFDTSNGNQAKNKQVIANGITYYFDTNGNGQYLFTNTGKSAVDDFTQRNAANSVNPSDYKNVVDGFFTADTWYRPKQILDNGTTWRNSNSNELRPMITAWWPNKDVQVNYLKLMQNNGLLDKSNSYSIQSDQQTLNQAAQKAQVNIEKKISQTGNTDWLNDLLFKGNGDNPSFVKQQYIWSSDSESPWQGDAWFQGGYLKYGNSVMTPNTNSNYRDSNNLFDFLLANDVDNSNPAVQAEDLNWLYYLTNFGTITANDSNANFDSIRIDAVDFISNDIIQRSYDYLRQKFNLMQSDANADSHISLVEGGVDAGTTSYSNDGLVEAPFRLDAYPLLHKQDGDVFKNLIDEEDSGIDISNHNGETNTNNTIGGITLSGGKPNYSIVHAHDKDVQEKVGQAIIDTTGIKDWTDFTPSQLAQGLETFYNDQRQTVKKYNDYNVPSAYAIMLTNKGTVPRIYYGDMYQDDGQFMQKKSLYYDDIANLMTARKKYVSGGQSMVDNNGILTSVRFGKGANTVSDSGTEDTRNQGIGLIVGSAPKKVLNDGDTVVLHMGAAHKNQKYRALMLTTENGIQNYNSDDNAPVAETDDNGDLVFSNKDINGQANTAIKQVANPEVNGYLAAWVPVGASDDQDSRTAPSTSQNNDGNVLHENDALDSNLIFEGFSNFQPTPTNHDEYANVVIAKNASLFKDWGVTSFEMAPQYRSSQDHTFVDSTIDNGYAFSDRYDLGFGTPTKYGTDEDLRNAIKSLHDNGMQVMADVVYNQLYNLPGQEVVSATRAGVTGNTNALPFGTQLYVVNTIGGGDYQKKYGGAFLNELQEQYPSLFKSQKYKYYYKNYANNGAGPGYLTVNDAERSDIPYNQPITEWSAKYMNGTNILGRGMGYVLKDWNTGDYFKLSGSDSTLPSSLTYKSGWVENPDSTWSYYEKNNIDKLTGSQVINEERVFFDNNGIQVKGGWVKNSNGTYSYYDKNSGNILTGDQLIDGEHFFFDNNGVQVKGKWIKNSDGSKSYYDSHLGKLIKTDKKVSSNARKKKSKEELLYENALKVLRKDKKRLDKNKTKANIRKYNKSLKKYRKAKKKLLAITKNRVANARKAIKIAKKVLSKRKNINNEKRYYKALKEYYVAEKSYLKITGNYNKKYYYEFDKLTPKVKVVKNIYSYKSRHFTKKNRVKKIKKGTLVRVKSIVRSGKVARINIGNGHFITSSKDFIKMFK

SEQ ID NO: 6 - protein, 1232 aa, Leuconostoc mesenteroides
  REGION 1..1232: note = MISC_FEATURE - BRS-E_delta 1
QQNNATLQVSPTKNDNSVAKNTTSTANVAKVDITTDNTRDISSANNVNNLITNQYKENSNGSWSYYDNNGQIVKGLQTINGNIQYFDSTTGEQVKGQTLTIDGVIYSFDKDSGNGTKTEVASLPTTGSYATKDGSNWQYEDQQQQPIKGLYTDKGNLRYFNETDGTQVKGTVVSVDNNTYYFDKDSGNGQLVPSVTGGQYGTIQLNNQTVWVYRNANGEIVKGLQNINGNIQYFDPNTGEQLKGKVATVNGVTYYFEASDGNLVGTVSDGLVTVNGQIQYFDPATGEQAKNKQVIVNNVTYYFDDNGYGQYLFTNAILSTTPDAYSAHTQAYNTDQSSFTNVVDGFLTADSWYRPKEVIADATGSAWQTSSENDYRPIITVWWPNKNVEVNYLKLMQDNDLLSTQTQFTIFSDQYTLNEAAQAAQNEIEKRIYREKSTDWLKDLLFEAHGDTPSFVKQQFIWNKDSEYQGAGGGNLWSLQGGYLKYVNDSETSWSDSTSRKHDYYEYLLGNDIDNSNPQVIAENINWLYYLMNFGSLTGNDDTANFDGVRMDAVIYMKGEASTKVYQFLHKSDELTKNEKIANEHISIVEDGTDETTKNNSALIVSKWNENIASSLAKIASGKDTSLEALVKTDEKTSVSRIMNSSVESDVNPNYSMIRSHDRGSQDEVINASKVANNDQSIALDNINLNQLENGLKLYYEDQASPTKNYNYYNIPASYALLLSNKDTVPRLYYSDMYQDYDQNPDTPQQYMSKPTIYYSAIDALLKARIKYVAGGQSMAVEKVGDNKDQEVLTSVRYGKNVMTATDTGTVESRTEGMGVIVSNNTKLKLSTTDQIVLHMGAAHANQAYQALMLTNDEGIQLYNDNAPVVWTDHNGDLVFNGNDINGQKNTSIKGYFNPQVAGYLAVWVPVGATDTQDARTKASTNATTDGKVFHSNAALDSNVIYEGFSNFQPIARNHNDFSNVKIAENVDLFKKWGITSFELAPQYRSADVSDLVGSTFVDVVTKNGYGLSDRYDLGFVTPTKYGSDSDLRNAISSLHAQGIQAMADFVGNQIYALNDGQEVVTAQRSDMFNNTLNNAFGTELYVVNSIGGGKYQAKYGGNYLEEIASLYPDLFTNQDGTKIDINTKIKQWSAKYMNGTNVLGRGMGYVLKDWNTATYFKLDGEHTVLPAALTLSGLKVENGVTYYYKNNERQTGTQTVDDVTYFFDPKTGAMKKDYFDFTADNKVYYYG

SEQ ID NO: 7 - protein, 1108 aa, Leuconostoc citreum
  REGION 1..1108: note = MISC_FEATURE - GBD-CD2 deltaN123 W2135L F2136L
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDALLQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 8 - protein, 1108 aa, Leuconostoc citreum
  REGION 1..1108: note = MISC_FEATURE - GBD-CD2 deltaN123 W2135L
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDALFQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 9 - protein, 1108 aa, Leuconostoc citreum
  REGION 1..1108: note = MISC_FEATURE - GBD-CD2 deltaN123 W2135I F2136Y
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDAIYQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 10 - protein, 1108 aa, Leuconostoc citreum
  REGION 1..1108: note = MISC_FEATURE - GBD-CD2 deltaN123 W2135I F2136C
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDAICQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 11 - protein, 1108 aa, Leuconostoc citreum
  REGION 1..1108: note = MISC_FEATURE - GBD-CD2 deltaN123 W2135V
AQAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDAVFQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLDGDKSTLPQIKGELKLEGKPIPNPLLGLDSTRTGHHHHHH

SEQ ID NO: 12 - protein, 1269 aa, Oenococcus kitaharae
  REGION 1..1269: note = MISC_FEATURE - DSR-OK
YYFDQDGHMRKGYSGVFDNQVLYFDKTTGALANTNVSSIKEGLTAQNDDFTAHNAVYSTKSESFTNIDGYLTAEAWYRPADILENGTDWRASRADEFRPILTTWWPDKQTEVNYLNYMKTQGFITNDQDFKLSDDQLLLNHAAQSVQGEIEKKISQQGSTDWLKTLLQTFINQQPSWNGESEDPGSDHLQGGALTFVNSPLTPDSNSNFRLLNRTPTNQTGTPQYDTDASLGGFELLLANDVDNSNPVVQAEQLNWLYYLLNFGSITADDPNANFDGIRIDAVDNVDADLLQIAAAYFKDAFKSGSNDQTTNQHLSILEDWSHNDPEYMKAQGYPQLTMDDYMHTQLIWSLTKPDNIRGTMQRFMDYYLVNRANDSTNNEAVANYSFVRAHDSEVQTVIAQIISDLYPNSGSGLIPTTDQLQAAFEVYNADMKSDVKKYTQYNIPSAYAMLLTNKDTVPRVYYGDMYTDDGDYMANKSPYFDAISTLLKARVKYAAGGQSMAVDKNDILTSVRFGQNAMLASDSGDNQTRQEGIGVIVSNNSHLKLAENDQVVLHMGAAHKNQAFRALLLTIESGLENFDTDLQAPVKYTDANGDLIFTAAELAGYLNPEVSGYLSAWVPVGAADNQDARTAADSATSTDGNVFHSNAALDSNVIFEGFSNFQSIPTAEQHDDFTNVKIAENAGLFKDWGITSFQLAPQYRSSTDSTFLDSIIQNGYAFTDRYDLGFDTPTKYGDVDDLRAAIKALHANNIQVMADWVPDQIYNLQNPEIITVNRTDSYGQPIAGSDLQNDLYLAYTNGGGQYQTKFGGAFLEKLQQLYPDLFTKTQISTGQTIDPSQKITEWSAKYFNGSNIQGRGAYYVLRDSGTDQYFKVISNDENEAFLPKQLTNQPGETGFSQDDQGIIFFSTSGYQAKNAFVQGDDGNYYYFDNTGHMVTGPQTINGRHYLFFPNGVEAQNVFVQNDRGETYYYDQRGRQVANQYVTDTNGNSFRFDENGIMLANQLAQVDGHWQFFKSSGVQAKDAFILGSDGKLRYFESGNGNMAVNEFKGSENGRYYYFGADGQAVSGLQTINGRQLYFDDHGQQMKDAFYTNQSGQRFYFNALTGDLVKGNFIYTSASSSFTPDNDSSDSYQGDSHLWYYADSQGQIVTGFQTINGHLQYFDDISGQMITNRFMRRADGNWIYLDENGEAVRGMRVINGLTNYFRDDFTQVKDGFAQDPNSGERHYFNGTNGAMVTNDYFSPDQIHWYYADDSGQPVTGFQTIKGQVQY

SEQ ID NO: 13 - protein, 1278 aa, Leuconostoc citreum
  REGION 1..1278: note = MISC_FEATURE - DSR-DP
MGKQLGWNTNKDGTIQYYVDQTNYLTGLTSLPVNPNLFSNKNSTPGYLFYDGRVVKQSTDFSSEFIMIDTKYSQITISSFDDNIQTGRIALCFYDSNQAFISRPLVGGSYTPFSNNYRNIYWKSVDIPTNAAYFRVSFGGDTIKQNSIKKVEFGNAITNWVAASGDDDTVIDNEYAKMMYYFDNSGFLQKSGNAIINGDKYYFDSKNGVVINGSKPQYDIGLTVISDEYTPHNKLYSTNAIDIENVDGFLTADSWYRPVDILDNGTTWRPSNKGEMRPVLMVWMPDKQTLVNYLNYMTSINMIGYSKTFTLKDNIKELLINQRQIQIAIEKKISQTGSTDFLRDVFSKFVATQDNWNKKSEYNDGDHLQGGALRFVNDDNLPKTNSSHRLFNRYPLNQKGGQNKDIGWNGGYELLLANDIDNSNPVVQAEQLNWLYFLTHLGSINSNDDTANFDGIRVDAVDNVDADLLQIAGDYYNETYGLNKDPNKANQKISILEDWAWGDYDYMGRMGQTQLTMDTGVKDGLMGALGYWNESVKPLETIISSMHTDRRKAFGNNRVVANYSFIRAHDAESQDIFSNMISANVKGADGWHPTWEQVEEGFKFYLADMESTLKLKTRNNIPAMYGILLTNKDTTPRVYYGDVFHEAGQYMANKTIYAPVIEKMLKYRITTVSGGQYMNMNNNALVSVRLGKYIDTLDTDYSKLTANQQKSARESGSVVVISNNKDYNLNNNNLVIELGLAHAGQTFKALAETINTHYNENSTGKLDLDTEREKTTNEKGQLIFTETEVVGSTSPHHWGYFSMWVPKEATLDHDVREFVDKNKYSDNNVFHAGEVLDSHVIYESFSNFTALPTKPEERANVVIKNNIDYFKSLGITVFEFAPQYRSSTDTSFLDSCLQNGYAFSDRYDLGYDNSPTKYGTVEDLQNAIKSLHNAGIQAMADYVPDQIYNLSLPEIVATNRVNSYGDLRDTTRFQSLYYVAKTKSNGADYQARFGGKFLNIMLMTTPKIFDKLQISTGEKIYPYEQISQWSAKYMNGTNTLGKGAYYVLKDWSSDKYFNINGVNTELPAQLKGDYTQYGFVNTTSGEIQYYSMSGAMAKDTFIQDEKSNWYYFDSDGNMAHKKLTILGYDYYFTENGIELINSSFADSDGNIVYTNSTGKGISVYQVIYKNGLYVQANNPTVLANGYQMFKDNKKYLWFDNGRLFSGFKIVDGRFYFLYQGEKITSSFQNVAGVKFYVDQNGDTISGLKYSNNQLMDFGHDDSFWLKKVLSEPKPEFMN

SEQ ID NO: 14 - protein, 1299 aa, Leuconostoc mesenteroides
  REGION 1..1299: note = MISC_FEATURE - DSR-S vardel delta 4N
TQQVSGKYVEKDGSWYYYFDDGKNAKGLSTIDNNIQYFYESGKQAKGQYVTIDNQTYYFDKGSGDELTGLQSIDGNIVAFNDEGQQIFNQYYQSENGTTYYFDDKGHAATGIKNIEGKNYYFDNLGQLKKGFSGVIDGQIMTFDQETGQEVSNTTSEIKEGLTTQNTDYSEHNAAHGTDAEDFENIDGYLTASSWYRPTGILRNGTDWEPSTDTDFRPILSVWWPDKNTQVNYLNYMADLGFISNADSFETGDSQSLLNEASNYVQKSIEMKISAQQSTEWLKDAMAAFIVAQPQWNETSEDMSNDHLQNGALTYVNSPLTPDANSNFRLLNRTPTNQTGEQAYNLDNSKGGFELLLANDVDNSNPVVQAEQLNWLYYLMNFGTITANDADANFDGIRVDAVDNVDADLLQIAADYFKLAYGVDQNDATANQHLSILEDWSHNDPLYVTDQGSNQLTMDDYVHTQLIWSLTKSSDIRGTMQRFVDYYMVDRSNDSTENEAIPNYSFVRAHDSEVQTVIAQIVSDLYPDVENSLAPTTEQLAAAFKVYNEDEKLADKKYTQYNMASAYAMLLTNKDTVPRVYYGDLYTDDGQYMATKSPYYDAINTLLKARVQYVAGGQSMSVDSNDVLTSVRYGKDAMTASDTGTSETRTEGIGVIVSNNAELQLEDGHTVTLHMGAAHKNQAYRALLSTTADGLAYYDTDENAPVAYTDANGDLIFTNESIYGVQNPQVSGYLAVWVPVGAQQDQDARTASDTTTNTSDKVFHSNAALDSQVIYEGFSNFQAFATDSSEYTNVVIAQNADQFKQWGVTSFQLAPQYRSSTDTSFLDSIIQNGYAFTDRYDLGYGTPTKYGTADQLRDAIKALHASGIQAIADWVPDQIYNLPEQELATVTRTNSFGDDDTDSDIDNALYVVQSRGGGQYQEMYGGAFLEELQALYPSLFKVNQISTGVPIDGSVKITEWAAKYFNGSNIQGKGAGYVLKDMGSNKYFKVVSNTEDGDYLPKQLTNDLSETGFTHDDKGIIYYTLSGYRAQNAFIQDDDNNYYYFDKTGHLVTGLQKINNHTYFFLPNGIELVKSFLQNEDGTIVYFDKKGHQVFDQYITDQNGNAYYFDDAGVMLKSGLATIDGHQQYFDQNGVQVKDKFVIGTDGYKYYFEPGSGNLAILRYVQNSKNQWFYFDGNGHAVTGFQTINGKKQYFYNDGHQSKGEFIDADGDTFYTSATDGRLVTGVQKINGITYAFDNTGNLITNQYYQLADGKYMLLDDSGRAKTGFVLQDGVLRYFDQNGEQVKDAIIVDPDTNLS

SEQ ID NO: 15 - protein, 1425 aa, Leuconostoc mesenteroides
  REGION 1..1425: note = MISC_FEATURE - ASR Cdelbis-deltathio
MKQQETVTRKKLYKSGKVWVAAATAFAVLGVSTVTTVHADTNSNVAVKQINNTGTNDSGEKKVPVPSTNNDSLKQGTDGFWYDSDGNRVDQKTNQILLTAEQLKKNNEKNLSVISDDTSKKDDENISKQTKIANQQTVDTAKGLTTSNLSDPITGGHYENHNGYFVYIDASGKQVTGLQNIDGNLQYFDDNGYQVKGSFRDVNGKHIYFDSVTGKASSNVDIVNGKAQGYDAQGNQLKKSYVADSSGQTYYFDGNGQPLIGLQTIDGNLQYFNQQGVQIKGGFQDVNNKRIYFAPNTGNAVANTEIINGKLQGRDANGNQVKNAFSKDVAGNTFYFDANGVMLTGLQTISGKTYYLDEQGHLRKNYAGTFNNQFMYFDADTGAGKTAIEYQFDQGLVSQSNENTPHNAAKSYDKSSFENVDGYLTADTWYRPTDILKNGDTWTASTETDMRPLLMTWWPDKQTQANYLNFMSSKGLGITTTYTAATSQKTLNDAAFVIQTAIEQQISLKKSTEWLRDAIDSFVKTQANWNKQTEDEAFDGLQWLQGGFLAYQDDSHRTPNTDSGNNRKLGRQPINIDGSKDTTDGKGSEFLLANDIDNSNPIVQAEQLNWLHYLMNFGSITGNNDNANFDGIRVDAVDNVDADLLKIAGDYFKALYGTDKSDANANKHLSILEDWNGKDPQYVNQQGNAQLTMDYTVTSQFGNSLTHGANNRSNMWYFLDTGYYLNGDLNKKIVDKNRPNSGTLVNRIANSGDTKVIPNYSFVRAHDYDAQDPIRKAMIDHGIIKNMQDTFTFDQLAQGMEFYYKDQENPSGFKKYNDYNLPSAYAMLLTNKDTVPRVYYGDMYLEGGQYMEKGTIYNPVISALLKARIKYVSGGQTMATDSSGKDLKDGETDLLTSVRFGKGIMTSDQTTTQDNSQDYKNQGIGVIVGNNPDLKLNNDKTITLHMGKAHKNQLYRALVLSNDSGIDVYDSDDKAPTLRTNDNGDLIFHKTNTFVKQDGTIINYEMKGSLNALISGYLGVWVPVGASDSQDARTVATESSSSNDGSVFHSNAALDSNVIYEGFSNFQAMPTSPEQSTNVVIATKANLFKELGITSFELAPQYRSSGDTNYGGMSFLDSFLNNGYAFTDRYDLGFNKADGNPNPTKYGTDQDLRNAIEALHKNGMQAIADWVPDQIYALPGKEVVTATRVDERGNQLKDTDFVNLLYVANTKSSGVDYQAKYGGEFLDKLREEYPSLFKQNQVSTGQPIDASTKIKQWSAKYMNGTNILHRGAYYVLKDWATNQYFNIAKTNEVFLPLQLQNKDAQTGFISDASGVKYYSISGYQAKDTFIEDGNGNWYYFDKDGYMVRSQQGENPIRTVETSVNTRNGNYYFMPNGVELRKGFGTDNSGNVYYFDDQGKMVRDKYINDDANNFYHLNVDGTMSRG

SEQ ID NO: 16 - protein, 1455 aa, Streptococcus mutans
  REGION 1..1455: note = MISC_FEATURE - GTF-SI
MEKKVRFKLRKVKKRWVTVSVASAVVTLTSLSGSLVKADSTDDRQQAVTESQASLVTTSEAAKETLTATDTSTATSATSQPTATVTDNVSTTNQSTNTTANTANFDVKPTTTSEQSKTDNSDKIIATSKAVNRLTATGKFVPANNNTAHSRTVTDKIVPIKPKIGKLKQPSSLSQDDIAALGNVKNIRKVNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRIHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGTRVVNGQRLYFKSNGVQAKGELITERKGRIKYYDPNSGNEVRNRYVRTSSGNWYYFGNDGYALIGWHVVEGRRVYFDENGVYRYASHDQRNHWDYDYRRDFGRGSSSAVRFRHSRNGFFDNFFRF

SEQ ID NO: 17 - protein, 1518 aa, Streptococcus salivarius
  REGION 1..1518: note = MISC_FEATURE - GTF-J
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 18 - protein, 1391 aa, Leuconostoc citreum
  REGION 1..1391: note = MISC_FEATURE - DSR-M delta 1
TPVGTTQSQQDLTGQTGQDKPTTKEVIDKKEPVPRVSAQNAGDLSADAKTTKADDKQDTQPTNAQLPDQGNKQTNSNSDKGVKESTTAPVKTTDVPSKSVAPETNTSINASDAISKSQEKQFEKAPDSVPETITGGRYSLKDGYYVYLDKQGKQVVGPKNIDNHLQYFDETTGKQVKGDFRSVNGKRIYFNANLGYADDYTTDVAGKLVGYDSNGNQVKAGYVTNSQGKTYYFNNQGEAIIGLKTDNNKTQYFGPDGAQVKGAFQQVNGKNIYFDAQTGYARQNVGFLDGTAKGFDEQGNQIKSGIATDLSGNVYYFDASGKMLTGVQNIDGKKYYFDEQGHRRRNYAGVFNNEFIYFGLDGVGQSAIEYQFEKGLTSQNSVATSHNAAKSYDTKSFTNVDGFLTANSWYRPTDILRNGTKWEPSTETDFRPLLMTWWPDKEVQANYLNYMSALGLGDQKIYTGASSQLDLNNAALIVQEAIEKKISLEKSTKWLDDSIKSFIKSKRKDIQGNLVDTNPGWTIDSETGSTNHLQNGAFIFTNSPLVPEANAAEGNRLINRTPSQQTGNHISYASQPYSGDDWGYELLLGNDVDNSNPIVQAEQLNWIHYLMNFGTITAPQDPDAHLANFDSIRIDAVDNVDADLLQIAGDYFKAAYQVGENDKNANQHIHILEDWSPNDVWYNQQVNGNSQLTMDATMQNQLLASLTRPITSRDSMKSFTKDALLVHRTADNSYNQAVPNYSFIRAHDSEVQTIIAKIISDKHPDLYPTVDKALLAKDSALYDEAFTEYNADMQKISSQKQYTHNNMPSAYAILLTNKDTVPRVYYGDLFTDNGEYMANKTPYYDAITSLLTARTKFVSGGQSLSVDKNDVLTSVRYGKGALSATDNGSSDTRNQGIGVIVSNNPNLDLNNDKVTLSMGISHAHQAYRPLLLTNSQGIVAYATDSEVPQNLYKTTNDKGELTFDASEIKGYDTVQTSGYLAVWVPVGASDEQDARTIASTEKNNGNSVYHSNAALDSQLIYEGFSNFQTVPSKNASADEYANVIIAKHAADFNKWGVTSFQMAPQYRSSTDGSFLDAVDTVQNGYAFTDRYDLGFNAADGSKNPTKYGTDEDLRNAIKSLHAQKTYDGSSIQVMADFVPDQLYNMPLEQAVSVIRTDKYGVNSENPDIQNIIYAANIKSSGTDYQSIYGGKYLAELQKNPLFKSLFDRIQISTKKTIDPNTRITQWSAKYFNGSNIQGKGINYVLKDWASNKYFNVSSNDDMYSRLPKQLMNQESNTGFIVDDIGVKYYSISGYQAKNTFVEDGNGEWYYFDNDGYMVKSTEESGPLRTVNASSKKYYILPNGVEIRNSFGQDIQGNTYYFDARGEMVTSQYISDDTQNIYYFNNDGTMAK

(19) United States
(12) Patent Application Publication
(10) Pub. No.: US 2025/0305017 A1
(43) Pub. Date: Oct. 2, 2025

(54) METHOD FOR PREPARING GLYCOLIPIDS, GLYCOLIPIDS AND USES THEREOF

(71) Applicants: Institut National de Recherche Pour |'Agriculture, l’Alimentation et |’Environnement, Paris (FR); Centre National de la Recherche Scientifique(CNRS), Paris (FR); Institut National des Sciences Appliquees de Toulouse, Toulouse Cedex 4 (FR); Universite de Bordeaux, Bordeaux (FR); Institut Polytechnique de Bordeaux, Talence (FR)

(72) Inventors: Etienne SEVERAC, Pibrac (FR); Magali REMAUD-SIMEON, Ramonville (FR); David GUIEYSSE, Montbrun-Lauragais (FR); Claire MOULIS, Villenouvelle (FR); Didier PINTORI, Leognan (FR); Vanessa KOMOROWSKI, Pessac (FR); Chrystel FAURE, Bordeaux (FR); Fernando LEAL CALDERON, La Brede (FR)

(73) Assignees: Institut National de Recherche Pour |'Agriculture, l’Alimentation et l’Environnement, Paris (FR); Centre National de la Recherche Scientifique (CNRS), Paris (FR); Institut National des Sciences Appliquees de Toulouse, Toulouse Cedex 4 (FR); Universite de Bordeaux, Bordeaux (FR); Institut Polytechnique de Bordeaux, Talence (FR)

(21) Appl. No.: 18/866,125

(22) PCT Filed: May 17, 2023
(86) PCT No.: PCT/FR2023/050717

§ 371 Date: Nov. 15, 2024

(30) Foreign Application Priority Data
May 18, 2022 (FR) FR2204730

(51) Int. Cl. C12P 19/44; A01N 25/04; A01N 25/30; A01P 1/00; C12N 9/10

(52) U.S. Cl. CPC C12P 19/44; A01N 25/04; A01N 25/30; A01P 1/00; C12N 9/1051

(57) ABSTRACT

Methods for preparing at least one glycolipid of formula (I): [Glc]n-xOy-R (I), with [Glc]n representing an osidic motif comprising 1-7 n glucosyl units; R representing a fatty acid radical with 4-24 carbon atoms; -xOy- symbolizing the attachment of the fatty acid radical to the osidic motif; the method comprising glucosylating a hydroxylated fatty acid of formula (II): R-yOH (II), where -yOH is a hydroxyl group attached to a carbon atom Cy; bringing the hydroxylated fatty acid of formula (II) into contact with at least one α-transglucosylase of the GH70 family in the presence of saccharose or a saccharose analogue.

The present invention relates to the field of enzymatic preparation of glycolipids. The present invention also relates to glycolipids and uses thereof.

Glycolipids are compounds comprising a saccharide part and a lipid part. Glycolipids can be of natural or biosynthetic origin. Glycolipids are interesting due to their amphiphilic character and their solubility properties.

Naturally occurring glycolipids are predominantly sophorolipids, consisting of a sophorose entity (glucose disaccharide with β-1,2 linkage) and a C16 or C18 fatty acid chain with unsaturation. Another category of natural glycolipids is represented by rhamnolipids consisting of one or two rhamnosyl units bonded to a fatty tail of 3-(hydroxyalkanoyloxy)-alkanoic acid (mainly C10 fatty acids).

Synthetic glycolipids are predominantly carbohydrate esters (glucose, sucrose, maltose), also called sucrose esters, in which the saccharide part and the lipid part are linked by an ester bond, or alkyl-(poly-)glucosides, also called alkyl polyglucosides, in which the saccharide part and the lipid part are linked by an ether bond.

Currently, glycolipids are conventionally synthesized via chemical routes. For example, the most common procedures for the synthesis of alkyl-(poly-)glycosides include the Fischer, Koenig-Knorr or Schmidt methods.

The Fischer method is the most widely used in industry. It allows the production of short chain alkyl polyglucosides in a single step. The carbohydrate is solubilized in excess alcohol in the presence of an acid catalyst (sulphonic acid, hydrochloric acid) and at elevated temperature (von Rybinski and Hill, Angew. Chem. Int. Ed. 37, 1328-1345, 1998). Obtaining larger APGs requires an additional step in which a small alkyl (generally butyl) is exchanged with a higher alcohol, making it possible to avoid reactant solubility problems.

This synthesis method is efficient but acid catalysts are difficult to recycle. Moreover, it is difficult to apply to obtaining di- or tri-glycosylated derivatives because of a competitive alcoholysis reaction of interglycosidic bonds (Koto et al., Simple preparations of alkyl and cycloalkyl α-glycosides of maltose, cellobiose and lactose, Carbohydr. Res. 339, 2415-2424, 2004).

The chemical synthesis of glycolipids of the alkyl polyglucoside type therefore involves a multitude of reaction steps including carbohydrate protection/deprotection steps; the use of acid, alkali or metal catalysts and large quantities of solvents to be recycled; risks of secondary reactions due to the reactivity of the catalysts and drastic synthesis conditions; the formation of two anomers in glycosylation products which are difficult to separate.

Ether-linked glycolipids can be biosynthesized enzymatically. The most studied enzymes in this context are those of the β-glycosidase family. These are cofactor-free enzymes that naturally hydrolyze glycosidic bonds present in polysaccharides to produce mono- or oligosaccharides. In vitro, these enzymes are able to use a glycosyl donor and catalyze the transfer of glycosyl to a free hydroxyl of an acceptor molecule and thus catalyze the formation of an ether bond between the acceptor molecule and the glycosyl residue. The synthesis of alkyl polyglucosides catalyzed by β-glycosidases generally conserves a bond of β configuration between the sugars and the alkyl part (β-alkyl polyglucosides).

This synthesis is possible either by hydrolysis reversal or by transglycosylation.

In the hydrolysis reversal approach, the glycosyl donors are polysaccharides or carbohydrates such as starch, cellulose, sucrose or glucose. In this case, the thermodynamic equilibrium is rather favorable to hydrolysis, so the yields of alkyl polyglucosides are generally very modest. For example, several teams working on almond β-glucosidase obtained production yields of less than 62% with short-chain alcohols (i.e. methylglucoside/xyloside or ethylglucoside/xyloside). The yields with alcohols of longer chains (carbon number greater than 6) are even lower and between 1 and 13% (Drouet et al., Biotechnol. Bioeng. 43, 1075-1080, 1994; Vic et al., Enzyme Microb. Technol. 20, 597-603, 1997; Yan et Liau, Biotechnol. Lett. 20, 653-657, 1998).

Other families of enzymes have made it possible to obtain alkyl polyglycosides.

Bousquet et al. demonstrated the possibility of obtaining α-alkyl polyglucosides using α-transglucosidase from Aspergillus niger (Bousquet et al., Bucke, C. (Ed.), Carbohydrate Biotechnology Protocols. Humana Press, Totowa, NJ, 291-296, 1999) or from Talaromyces duponti (Bousquet et al., Enzyme Microb. Technol. 23, 83-90, 1998). Thus, α-butyl-glucoside was obtained by transfer reaction of glucosyl units derived from maltodextrins on butanol in a biphasic medium.

Dahiya et al. were able to produce 1-O-alkylyl-α-D-mono, di- and tri-glucopyranosides (α-alkyl polyglycosides) from hexanol or octanol and sucrose using a Microbacterium paraoxydans strain exhibiting amylosucrase membrane transglycosylation activity with a maximum yield of 14.8% (Dahiya et al., Biotechnol. Lett. 37, 1431-1437, 2015).

Ochs et al., 2011, have shown the possibility of producing pentyl- and octyl-polyxylosides (alkyl-polyxylosides) by transglycosylation reaction between pentanol or octanol and xylans from birch wood catalyzed by different xylanases including that of Thermobacillus xylanaticus (Ochs et al., Green Chem. 13, 2380-2388, 2011; Remond et al., FR2967164, 2012).

Some glucansucrases (GS) from the 13 and 70 families of glycoside hydrolases (GH13 and GH70) have been used in acceptor reactions to lengthen the carbohydrate portion of short alkyl monoglucosides (1 to 8 carbon atoms) from sucrose.

GH70 glucansucrases have also been used in direct glucosylation reactions of alcohols. Indeed, Seibel et al. have shown the ability of dextransucrase from Streptococcus oralis GTFR to glucosylate alcohols ranging from (chloro) methanol to (4-chloro) 1-butanol (Seibel et al., ChemBioChem 7, 310-320, 2006). Moreover, Kim et al. have shown, during glucosylation tests of short alcohols of 1 to 4 carbon atoms, a preference of dextransucrase from L. mesenteroides B-1299CB for primary alcohols over secondary alcohols, tertiary alcohols having not been able to be converted (Kim et al., Biotechnol. Lett. 31, 1433-1438, 2009).

However, most known enzymatic methods are limited because they rarely allow glycolipids having carbon chains comprising more than 8 carbon atoms to be obtained.

There is therefore a need for an economical and simple method to implement on an industrial scale, which makes it possible to prepare glycolipids having new particular molecular architectures, in particular glycolipids with a bolaamphiphilic structure, which are likely to find applications in fields such as pharmacy, cosmetics, fine chemicals, biocontrol, plant protection products, decontamination or food processing.

### BRIEF DESCRIPTION OF THE FIGURES

FIG. 1 represents the screening of α-transglucosylases of the GH70 family for their ability to glucosylate 11-[(2-hydroxy-ethyl) sulfanyl)]-undecanoic acid (HESUA). Determination of the conversion rate of the lipid acceptor under the conditions tested: [HESUA]=10 mM, [sucrose]=292 mM, DMSO=10% v/v; 50 mM sodium acetate buffer pH=5.75, enzymatic activity=1 U·mL⁻¹, T=30° C.

FIG. 2 represents the screening of α-transglucosylases of the GH70 family for their ability to glucosylate 11-hydroxyundecanoic acid (HUDA). Determination of the conversion rate of the lipid acceptor under the conditions tested: [HUDA]=10 mM, [sucrose]=292 mM, DMSO=10% v/v; 50 mM sodium acetate buffer pH=5.75, enzymatic activity=1 U·mL⁻¹, T=30° C.

FIG. 3 represents the screening of α-transglucosylases of the GH70 family for their ability to glucosylate erythro-aleuritic acid (EAA). Determination of the conversion rate of the lipid acceptors under the conditions tested: [EAA]=10 mM; [sucrose]=292 mM; DMSO=10% v/v; 50 mM sodium acetate buffer pH=5.75, enzymatic activity=1 U·mL⁻¹, T=30° C.

FIG. 4 illustrates a comparison of glucosylation product profiles of 11-[(2-hydroxy-ethyl) sulfanyl)]-undecanoic acid (HESUA) by branching enzymes BRS-A, BRS-B Δ1, BRS-C, BRS-D Δ1, BRS-E and GBD-CD2 ΔN123. Reactions carried out at 30° C. under 800 rpm stirring in the presence of 438.5 mM sucrose, 10 mM HESUA, 10% v/v DMSO and 50 mM sodium acetate buffer at pH=5.75. Numbers 1 to 6 represent the major glucosylation products. The enzymes are grouped according to their glycoside binding specificity on dextrans, i.e. α-1,2 for BRS-A, BRS-D Δ1, GBD-CD2 ΔN123 or α-1,3 for BRS-B Δ1, BRS-C, BRS-E Δ1).

FIG. 5 illustrates a comparison of glucosylation product profiles of 11-[(2-hydroxy-ethyl) sulfanyl)]-undecanoic acid (HUDA) by synthesized branching enzymes BRS-A, BRS-B Δ1, BRS-C, BRS-D Δ1, BRS-E Δ1 and GBD-CD2 ΔN123. Reactions carried out at 30° C. under 800 rpm stirring in the presence of 438.5 mM sucrose, 10 mM HUDA, 10% v/v DMSO and 50 mM sodium acetate buffer at pH=5.75. The enzymes are grouped according to their glycoside binding synthesis specificity on dextrans, i.e., α-1,2 for BRS-A, BRS-D Δ1, GBD-CD2 ΔN123 or α-1,3 for BRS-B Δ1, BRS-C, BRS-E Δ1).

FIG. 6 represents a comparison of the glucosylation product profiles of erythro-aleuritic acid (EAA) synthesized by the enzymes BRS-A, BRS-B Δ1, BRS-C, BRS-D Δ1, BRS-E Δ1 and GBD-CD2 ΔN123. Reactions carried out at 30° C. under 800 rpm stirring in the presence of 438.5 mM sucrose, 10 mM EAA, 10% v/v DMSO and 50 mM sodium acetate buffer at pH=5.75. Numbers 1 to 16 represent the peaks of the major glucosylation products. The enzymes are grouped according to their glycoside binding synthesis specificity on dextrans, i.e., α-1,2 for BRS-A, BRS-D Δ1, GBD-CD2 ΔN123 or α-1,3 for BRS-B Δ1, BRS-C, BRS-E Δ1).

FIG. 7 shows the conversion rate of 11-[(2-hydroxyethyl) sulfanyl)]-undecanoic acid (HESUA) obtained during its glucosylation by the branching enzymes BRS-A, BRS-B Δ1, BRS-C, BRS-D Δ1, BRS-E Δ1 and GBD-CD2 ΔN123 as a function of the initial sucrose concentration. Reactions carried out at 30° C. under 800 rpm stirring in the presence of increasing concentrations of sucrose; 10 mM 11-[(2-hydroxy-ethyl) sulfanyl)]-undecanoic acid; 10% v/v DMSO and 50 mM sodium acetate buffer at pH=5.75.

FIG. 8 represents the conversion rate of HUDA obtained during glucosylation by the branching enzymes BRS-A, BRS-B Δ1, BRS-C, BRS-D Δ1, BRS-E Δ1 and GBD-CD2 ΔN123 as a function of the initial sucrose concentration. Reactions carried out at 30° C. under 800 rpm stirring in the presence of increasing concentrations of sucrose, 10 mM HUDA, 10% v/v DMSO and 50 mM sodium acetate buffer at pH=5.75.

FIG. 9 represents the conversion rate of erythro-aleuritic acid (EAA) obtained during glucosylation by branching enzymes BRS-A, BRS-B Δ1, BRS-C, BRS-D Δ1, BRS-E Δ1 and GBD-CD2 ΔN123 as a function of the initial sucrose concentration. Reactions carried out at 30° C. under 800 rpm stirring in the presence of increasing concentrations of sucrose; 10 mM EAA; 10% v/v DMSO, 1 U·mL⁻¹ of enzymatic activity in reaction and 50 mM sodium acetate buffer at pH=5.75.

FIG. 10 represents a chromatogram of the glucosylation products of 11-[(2-hydroxy-ethyl) sulfanyl)]-undecanoic acid (HESUA) obtained with the enzyme BRS-A after removal of free sugars by chromatography on Purasorb PAD910 resin.

FIG. 11 represents a chromatogram of the glucosylation products of 11-hydroxyundecanoic acid (HUDA) after removal of free sugars by flash chromatography and mass spectrometric analysis of the purified products corresponding to peaks 1 and 2 (glucosylation products 1 and 2).

FIG. 12 represents a chromatogram of the glucosylation products of erythro-aleuritic acid (EAA) after pre-purification (removal of free sugars) by flash chromatography and mass spectrometric analysis of the purified products.

FIG. 13A FIG. 13B FIG. 13C represent the glucosylation profile of 12-hydroxydodecanoic acid (HDDA) ([FIG. 13A]), 15-hydroxypentadecanoic acid (HPDA) ([FIG. 13B]) and 16-hydroxy-hexadecanoic acid (HHDA) ([FIG. 13C]) by the branching enzymes BRS-A, BRS-B Δ1, BRS-C, BRS-D Δ1, BRS-E Δ1 and GBD-CD2 ΔN123.

FIG. 14A FIG. 14B represent the glucosylation profile of 9,10 dihydroxyoctanoic acid (DHOA) (FIG. 14A) and 9,10,12 trihydroxyoctadecanoic acid (THOA) (FIG. 14B) by the branching enzymes BRS-A, BRS-B Δ1, BRS-C, BRS-D Δ1, BRS-E Δ1 and GBD-CD2 ΔN123. Reactions carried out at 37° C. under 800 rpm stirring in the presence of 877 mM sucrose, 5 mM fatty acid, an enzymatic activity of 1 U·mL⁻¹, 10% v/v DMSO and 50 mM sodium acetate buffer at pH=5.75. The enzymes are grouped according to their glycoside binding synthesis specificity on dextrans, i.e., α-1,2 for BRS-A, BRS-D Δ1, GBD-CD2 ΔN123 or α-1,3 for BRS-B Δ1, BRS-C, BRS-E Δ1).

FIG. 15 illustrates the characterization at day 0 of emulsions consisting of an aqueous solution containing 2% Glc-HESUA and 20% by mass of dodecane, at the different pH values studied. D (4.3) represents the volume mean diameter and P the polydispersity.

FIG. 16 illustrates the stability monitoring over 14 days of emulsions whose aqueous phase comprises 2% by weight of Glc-HESUA (O/W 20/80). The size of the drops is determined by particle size analysis and confirmed by light microscopy.

FIG. 17: Study of the destabilization of the emulsion (2% Glc-HESUA in water/dodecane 80/20). Left: the emulsion is produced and maintained at pH=6. Right: the emulsion is produced at pH=6 and then adjusted to pH=2 using a 0.1 M HCl solution.

FIG. 18 illustrates the characterization at day 0 of the emulsions consisting of an aqueous solution containing 2% Glc-HUDA and 20% by mass of dodecane, at the different pH values studied. D (4.3) represents the volume mean diameter and P the polydispersity.

FIG. 19: Stability monitoring over several weeks of emulsions containing 2% surfactant (O/W 20/80). The size of the drops is determined by particle size analysis and confirmed by light microscopy.

FIG. 20 illustrates the destabilization of the emulsion (2% GHA in water/dodecane 80/20). Left: the emulsion is produced and maintained at pH=6. Right: the emulsion is produced at pH=6 and then adjusted to pH=2 using a 0.1 M HCl solution. GHA=glycosylated form of HEDA

FIG. 21 represents the influence of the hydrophobicity of the support, the nature of the oil, the evaporation, the size and the concentration of the drops on the intensity of the phenomena observed during the deposition of a drop of emulsion on a solid support.

FIG. 22 shows the macroscopic appearance of the primary emulsion (a), the macroscopic appearance of the dry emulsion (powder) (b), the microscopic observation of the emulsion before drying (c), the microscopic observation of the emulsion after drying and redispersion in water (d); the measurement of the distribution of the droplet size before drying and after drying and then redispersion in water (e).

FIG. 23 represents the monitoring of E. coli K12 cultures (OD 600 nm) in the presence of concentrations of 0% w/v (+), 0.5% w/v (□), 1% w/v (Δ), 2% w/v (×) and 2.5% w/v (*) of glucosylation products of HUDA (A) and EAA (B) in aqueous phase.

### BRIEF DESCRIPTION ACCORDING TO THE INVENTION

An aim of the present invention is to overcome the disadvantages of the prior art and to provide a method for preparing a glycolipid of formula (I) by enzymatic catalysis.

Surprisingly, the inventors have discovered that α-transglucosylases are capable of glucosylating hydroxy fatty acids.

An advantage of the method according to the invention is that it uses renewable and inexpensive substrates, i.e., sucrose or one of its analogs and bio-based hydroxy fatty acids.

The method also has the advantage that it makes it possible to obtain glycolipids having a bolaamphiphilic type structure (i.e., consisting of two polar parts separated by a hydrophobic part) which suggests interesting surfactant and biological properties. The glycolipids according to the invention are, however, distinguished from sophorolipids in that their saccharide motif may comprise a single glucosyl unit or else several glucosyl units linked by a bonds whose nature can be modulated (α-1,2 and/or α-1,3 and/or α-1,4 and/or α-1,6) as a function of the α-transglucosylase used.

Significantly, the method according to the invention makes it possible to prepare bolaamphiphilic glycolipids whose hydrophobic part comprises a carbon chain of large size, typically greater than 8 carbon atoms, which may be interrupted by at least one sulfanyl group, and substituted, for example, by at least one hydroxyl group.

The method according to the invention therefore makes it possible to obtain a great diversity of glycolipids in terms of size of their hydrophobic part and structure of their glucoside part.

This diversity is very advantageous for producing new, interesting glycolipids useful in fields such as pharmacy, cosmetics, fine chemistry, biocontrol, plant protection, decontamination or food processing.

Another aim according to the invention is to provide glycolipids, in particular bolaamphiphilic glycolipids with great diversity of molecular architecture, and which can be useful as a surfactant or as an antimicrobial agent, especially as an antibacterial agent.

These aims are achieved by the invention which will be described below.

Thus, an object according to the invention is a method for preparing at least one glycolipid corresponding to formula (I):

[Glc]ₙ-xOy-R (I)
wherein
[Glc]ₙ represents a linear or branched saccharide motif comprising n glucosyl units,
with n comprised between 1 and 7,
with the condition that, when the saccharide motif comprises several glucosyl units, these units are linked together by α-type glycosidic bonds;
R represents a fatty acid radical comprising between 4 and 24 carbon atoms, the carbon chain of which is linear or branched, saturated or unsaturated, optionally interrupted by one or more sulfur atoms, optionally also possibly comprising one or more substituent(s) chosen from a hydroxyl group, carbonyl group, methoxyl group, amine group, nitrosyl group and thiol group,
-xOy- symbolizes the attachment of the fatty acid radical to the saccharide motif by an ether bond linking a Cx carbon atom of a glucosyl residue of the saccharide motif, previously bearing a hydroxyl group, to a Cy carbon atom of the fatty acid radical, previously bearing a hydroxyl group, with Cx being the position of the atom of the glucosyl residue on which the bond is effected, Cx representing the C1 carbon atom of the glucosyl residue, Cy being a carbon atom positioned along the carbon chain of the fatty acid radical or at the omega end thereof;
said method comprising a step of glucosylation of a hydroxy fatty acid of formula (II):

R-yOH (II)
wherein
R is a fatty acid radical as defined in formula (I),
-yOH represents a hydroxyl group attached to a Cy carbon atom as defined above; said step comprising contacting the hydroxy fatty acid of formula (II) with at least one α-transglucosylase of the GH70 family in the presence of sucrose or a sucrose analog.

Another object according to the invention relates to a glycolipid corresponding to formula (I):

[Glc]ₙ-xOy-R (I)
wherein
[Glc]ₙ represents a linear or branched saccharide motif comprising n glucosyl units,
with n comprised between 1 and 7,
with the condition that, when the saccharide motif comprises several glucosyl units, these units are linked together by α-type glycosidic bonds;
R represents a fatty acid radical comprising between 4 and 24 carbon atoms, the carbon chain of which is linear or branched, saturated or unsaturated, optionally interrupted by one or more sulfur atoms, optionally also possibly comprising one or more substituent(s) chosen from a hydroxyl group, carbonyl group, methoxyl group, amine group, nitrosyl group and thiol group,
-xOy- symbolizes the attachment of the fatty acid radical to the saccharide motif by an ether bond linking a Cx carbon atom of a glucosyl residue of the saccharide motif, previously bearing a hydroxyl group, to a Cy carbon atom of the fatty acid radical, previously bearing a hydroxyl group, with Cx being the position of the atom of the glucosyl residue on which the bond is effected, Cx representing the C1 carbon atom of the glucosyl residue, Cy being a carbon atom positioned along the carbon chain of the fatty acid radical or at the omega end thereof.

Another object according to the invention concerns the use of a glycolipid according to the invention, as surfactant.

Another object according to the invention concerns the use of a glycolipid according to the invention as defined above, as an antimicrobial agent.

Another object according to the invention relates to an oil-in-water emulsion comprising an oil phase dispersed in an aqueous phase and at least one glycolipid of formula (I) according to the invention, characterized in that the emulsion is kinetically stable when the pH of the aqueous phase is greater than a threshold potential of hydrogen pHs advantageously comprised between 2 and 5.

Another object concerns the use of an emulsion according to the invention for cleaning surfaces.

### DETAILED DESCRIPTION ACCORDING TO THE INVENTION

### Method for Preparing at Least One Glycolipid According to the Invention

In the context of the present invention, the expressions “glycolipid of formula (I)”, “glycolipid” and “glycolipid according to the invention” can be used interchangeably. In this document, the terms “glycolipid” and “glucolipid” can be used interchangeably.

In the context of the present invention, the expressions “hydroxy fatty acid of formula (II)”, “hydroxy fatty acid” and “hydroxy fatty acid according to the invention” can be used interchangeably.

### Saccharide Motif

According to the invention, the expression “saccharide motif” denotes a linear or branched polymer consisting of glucosyl units linked together by glycosidic bonds.

According to the invention, the terms “glycosidic bond” or “glucosidic bond” can be used interchangeably and denotes a covalent bond that binds one glucosyl to another glucosyl adjacent within the saccharide motif.

The saccharide motif [Glc]ₙ corresponds to the n glucosyl units grafted during the glucosylation step at the level of the Cy carbon of the hydroxy fatty acid according to the invention by the α-transglucosylase of the GH70 family.

The number of glucosyl units in the saccharide motif [Glc]ₙ of the glycolipid according to the invention is represented by n. The value n is advantageously comprised between 1 and 7, preferably comprised between 1 and 4. Preferably, n is equal to 1 or 2.

In a particular embodiment, the glycolipid according to the invention is a glycolipid whose saccharide motif comprises several glucosyl units. Advantageously, the glycolipid according to the invention is such that n is between 2 and 7, preferably between 2 and 4.

Bonds within the Saccharide Motif

According to the invention, the expression “a bond” designates a covalent bond which binds the C1 carbon atom of a glucosyl unit in its α configuration; the expression “β bond” designates a covalent bond which binds the C1 carbon atom of a glucosyl unit in its β configuration.

When the saccharide motif comprises several glucosyl units, the latter are linked together by α-type glycosidic bonds. Advantageously, the choice of the α-transglucosylase type of the GH70 family makes it possible to modulate the type of binding within the saccharide motif.

The glycosidic bond(s) within the saccharide motif are advantageously chosen from an α-1,2 glycosidic bond, an α-1,3 bond, an α-1,4 bond or an α-1,6 bond, or a mixture thereof; more preferably chosen from an α-1,2 bond, an α-1,3 bond, an α-1,4 bond, an α-1,6 bond or a mixture thereof.

According to the invention, the term “α-1,3 bond” designates the covalent ether bond which binds the C1 carbon atom of a glucosyl unit formerly carrying the hemiacetal function in its α configuration and the hydroxyl carried by the C3 carbon of another adjacent glucosyl unit. According to the invention, the term “α-1,2 bond” designates the covalent ether bond which binds the C1 carbon atom of a glucosyl unit formerly carrying the hemiacetal function in its α configuration and the hydroxyl carried by the C2 carbon of another adjacent glucosyl unit. According to the invention, the term “α-1,4 bond” designates the covalent ether bond which binds the C1 carbon atom of a glucosyl unit formerly carrying the hemiacetal function in its α configuration and the hydroxyl carried by the C4 carbon of another adjacent glucosyl unit. According to the invention, the term “α-1,6 bond” designates the covalent ether bond which binds the C1 carbon atom of a glucosyl unit formerly carrying the hemiacetal function in its α configuration and the hydroxyl carried by the C6 carbon of another adjacent glucosyl unit.

The α-glucosyl units are preferably α-D-glucosyl units.

The glycosidic bonds can be analyzed by methods known to the person skilled in the art.

For example, the glycosidic bonds within the saccharide motif can be analyzed by nuclear magnetic resonance (NMR) or mass spectrometry (MS).

### Bond Between the Saccharide Motif and the Fatty Acid Radical

-xOy- symbolizes the attachment of the fatty acid radical to the saccharide motif by an ether bond linking a Cx carbon atom of a glucosyl residue of the saccharide motif, previously bearing a hemiacetal group, to a Cy carbon atom of the fatty acid radical, previously bearing a hydroxyl group, with Cx being the position of the atom of the glucosyl residue on which the bond is effected, Cx representing the C1 carbon atom of the glucosyl residue, Cy being a carbon atom positioned along the carbon chain of the fatty acid radical or at the omega end.

For the purposes of the present invention, the position of the carbon atoms of a glucosyl unit is numbered so that the C1 carbon atom of a glucosyl unit is the carbon atom carrying the aldehyde function —CHO in the open form of this glucosyl unit.

For the purposes of the present invention, the position of the carbon atoms of the fatty acid radical is numbered so that C′1 represents the carbon atom of the carboxylic acid function of the fatty acid radical.

For the purposes of the present invention, the omega end of the fatty acid radical is the end terminated by a carbon called omega carbon whose position is opposite to the carbon carrying the carboxylic acid group of the fatty acid radical.

In the glycolipid according to the invention, the glucosyl unit adjacent to the fatty acid radical is advantageously linked to the fatty acid radical by an α bond.

In other words, in the glycolipid of formula (I), the glucosyl unit adjacent to the fatty acid radical is preferably in the α configuration.

The bond between the saccharide motif and the fatty acid radical can be analyzed by any method known to the person skilled in the art, for example by nuclear magnetic resonance (NMR).

### Fatty Acid Radical

Advantageously, the fatty acid radical comprises at least 6, 7, 8, 9, 10 or 11 carbon atoms. Advantageously, the fatty acid radical comprises at most 24 carbon atoms. Advantageously, the fatty acid radical comprises between 6 and 24 carbon atoms, between 7 and 24 carbon atoms, between 8 and 24 carbon atoms, between 9 and 24 carbon atoms, between 10 and 24 carbon atoms, preferably between 11 and 24 carbon atoms, preferably between 11 and 20 carbon atoms.

According to a particular embodiment, the radical R can be represented by the following general formula:

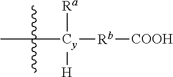

wherein
   the wavy line [symbol] represents the attachment point to -xOy-
   C_{y} is as defined above and
   R^{a} represents H or a carbon chain comprising between 1 and 22 carbon atoms, said chain being linear or branched, saturated or unsaturated, optionally interrupted by one or more sulfur atoms, optionally further comprising one or more substituents chosen from a hydroxyl group, carbonyl group, methoxyl group, amine group, nitrosyl group and thiol group,
   R^{b} represents a carbon chain comprising between 1 and 22 carbon atoms, said chain being linear or branched, saturated or unsaturated, optionally interrupted by one or more sulfur atoms, optionally also possibly comprising one or more substituent(s) chosen from a hydroxyl group, carbonyl group, methoxyl group, amine group, nitrosyl group and thiol group,
   provided that the sum of the carbon atoms comprised in the two radicals R^{a} and R^{b} is comprised between 2 and 22, and preferably between 4 and 22.

Preferably, R^{a} represents H (case where Cy is the carbon at the omega end of the fatty acid radical) or a linear carbon chain, optionally interrupted by one or more sulfur atoms, optionally further able to comprise one or more substituent(s) chosen from a hydroxyl group, carbonyl group, methoxyl group, amine group, nitrosyl group and thiol group (case where Cy is a carbon positioned along the carbon chain of the fatty acid radical) and R^{b} represents a linear carbon chain, optionally interrupted by one or more sulfur atoms, optionally further being able to comprise one or more substituent(s) chosen from a hydroxyl group, carbonyl group, methoxyl group, amine group, nitrosyl group, and thiol group.

According to a particular embodiment, the carbon chain of the fatty acid radical is linear, i.e., Cy is the carbon at the omega end of the fatty acid radical. In this embodiment, R^{a} represents H and R^{b} represents a linear carbon chain comprising between 2 and 22 carbon atoms, preferably between 4 and 22 carbon atoms, optionally interrupted by one or more sulfur atoms, optionally further comprising one or more substituents chosen from a hydroxyl group, carbonyl group, methoxyl group, amine group, nitrosyl group and thiol group.

According to a preferred embodiment, when the fatty acid radical comprises an amine substituent (NH or NH₂ group), this amine group is not located in the α position relative to the carboxyl functional group.

Preferably, the optional substituent(s) of the carbon chain are chosen from a hydroxyl group, carbonyl group, methoxyl group or thiol group, more preferably from a hydroxyl or thiol group.

Typically, the carbon chain (or radical) R optionally comprises at most three hydroxyl groups, preferably at most three substituents chosen from a hydroxyl group, carbonyl group, methoxyl group, amine group, nitrosyl group, and thiol group, preferably chosen from a hydroxyl group, carbonyl group, methoxyl group, and thiol group, preferably at most three substituents. More specifically:
when the carbon chain comprises between 4 and 11 carbon atoms (preferably between 6 and 11 carbon atoms), said carbon chain comprises at most one hydroxyl group, preferably at most one substituent chosen from a hydroxyl group, carbonyl group, methoxyl group, and thiol group, preferably at most one substituent;
when the carbon chain comprises between 12 and 16 carbon atoms, said carbon chain comprises at most two hydroxyl groups, preferably at most two substituents chosen from a hydroxyl group, carbonyl group, methoxyl group and thiol group, preferably at most two substituents;
when the carbon chain comprises between 17 and 24 carbon atoms, said carbon chain comprises at most three hydroxyl groups, preferably at most three substituents chosen from a hydroxyl group, carbonyl group, methoxyl group and thiol group, preferably at most three substituents.

In a particularly advantageous manner:
i) the radical R can be represented by the following general formula:

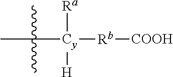

wherein the wavy line [symbol] , Cy, R^{a} and R^{b} are as defined above (any of the embodiments described above); and
ii) R^{a} and R^{b} together optionally comprise at most three hydroxyl groups (in other words, at most three hydroxyl groups are distributed over Rₐ and R_{b}), preferably at most three substituents chosen from a hydroxyl group, carbonyl group, methoxyl group, amine group, nitrosyl group and thiol group, preferably chosen from a hydroxyl group, carbonyl group, methoxyl group and thiol group, preferably at most three substituents; and
iii) when R^{a} or R^{b} comprise an amine substituent (NH or NH₂ group), this amine group is not located in the α position relative to the carboxyl functional group.

In this particularly preferred embodiment, feature ii) is preferably such that:
When R^{a} and R^{b} between them comprise between 2 and 9 carbon atoms (preferably between 4 and 9 carbon atoms), R^{a} and R^{b} between them comprise at most one hydroxyl group, preferably at most one substituent chosen from a hydroxyl group, carbonyl group, methoxyl group, and thiol group, preferably at most one substituent;
when R^{a} and R^{b} between them comprise between 10 and 14 carbon atoms, R^{a} and R^{b} comprise between them at most two hydroxyl groups, preferably at most two substituents chosen from a hydroxyl group, carbonyl group, methoxyl group and thiol group, preferably at most two substituents;
when R^{a} and R^{b} between them comprise between 15 and 22 carbon atoms, R^{a} and R^{b} between them comprise at most three hydroxyl groups, preferably at most three substituents chosen from a hydroxyl group, carbonyl group, methoxyl group and thiol group, preferably at most three substituents.

### Glycolipid Mixture

In one embodiment, the method according to the invention makes it possible to prepare a mixture of glycolipids according to the invention. Advantageously, the mixture contains glycolipids according to the invention which differ only in the number of glucosyl units of their saccharide motif and possibly the nature of the glycosidic bond.

Preferably, the mixture consists essentially of glycolipids in which n is comprised between 1 and 7, preferably n is comprised between 1 and 4, preferably in which n is equal to 1, 2 or 3, more preferably in which n is equal to 1 or 2. Preferably, the mixture of glycolipids according to the invention comprises from 70% to 100%, preferably from 90% to 100% by weight of glycolipids according to the invention in which n is comprised between 1 and 7, preferably n is comprised between 1 and 4, preferably in which n is equal to 1, 2 or 3, more preferably in which n is equal to 1 or 2, in % by weight relative to the total weight of glycolipids of the mixture.

### Hydroxy Fatty Acids

The method according to the invention is carried out with a hydroxy fatty acid of formula (II)

R-yOH (II)
wherein
R is a fatty acid radical as defined in formula (I),
-yOH represents a hydroxyl group attached to a Cy carbon atom as defined above.

The method can especially be carried out with three categories of hydroxy fatty acids.

### First Variant: Sulfanylated Hydroxy Fatty Acid

According to a first variant, the method according to the invention is carried out with a sulfanylated hydroxy fatty acid.

In this embodiment, the hydroxy fatty acid advantageously corresponds to formula (IIA):

HO—(CH₂)_{b}—S—(CH₂)ₐ—COOH (IIA)
wherein
a is comprised between 9 and 14,
b is greater than or equal to 2, and
with the sum of a and b less than 23.

The hydroxy fatty acid of formula (IIB) is, for example, 11-[(2-hydroxyethyl) sulfanyl] undecanoic acid OH—(CH₂)₂—S—(CH₂)₁₀—COOH.

### Second Variant: Fatty Acid Hydroxylated in the Omega Position

According to a second variant, the method according to the invention is carried out with a fatty acid hydroxylated in the omega position.

In one embodiment, the hydroxy fatty acid corresponds to formula (IIB):

OH—(CH₂)ₐ—COOH (IIB)

wherein a is comprised between 3 and 23, preferably between 10 and 15.

Advantageously, a is comprised between 11 and 15.

The hydroxy fatty acid of formula (IIB) is, for example, 11-hydroxyundecanoic acid OH—(CH₂)₁₀—COOH.

### Third Variant: Polyhydroxy Fatty Acids

According to a third variant, the method according to the invention is carried out with a polyhydroxy fatty acid, preferably chosen from a dihydroxy fatty acid and a trihydroxy fatty acid.

Preferably, the dihydroxy fatty acid is:
a dihydroxy fatty acid whose carbon chain is substituted by two hydroxyl groups positioned along the carbon chain, for example 9,10-dihydroxy octadecanoic acid, or
a dihydroxy fatty acid whose carbon chain is substituted by a hydroxyl group positioned along the chain and a hydroxyl group positioned at the omega end thereof.

Advantageously, the trihydroxy fatty acid is:
a trihydroxy fatty acid whose carbon chain is substituted by two hydroxyl groups positioned along the carbon chain and by a hydroxyl group in the omega position of the carbon chain, for example erythro-aleuritic acid, or
a trihydroxy fatty acid whose carbon chain is substituted by three groups positioned along the carbon chain, for example 9,10,12-trihydroxyoctadecanoic acid or 9,10,12-trihydroxystearic acid.

### α-Transglucosylase

The inventors have, for the first time, shown that GH70 family α-transglucosylases are capable of catalyzing the glucosylation of hydroxy fatty acids from sucrose.

The inventors have also shown that, unexpectedly, this glucosylation is particularly efficient when the α-transglucosylases of the GH70 family used are branching sucrases of the GH70 family.

According to the invention, the term “α-transglucosylase” designates an enzyme capable of polymerizing glucosyl units according to a bonds, by catalyzing the transfer of a glucosyl unit from a glucosyl donor sugar to a hydroxylated acceptor compound.

According to the invention, the expression “of the GH70 family”, relating to α-transglucosylase, means that the α-transglucosylase according to the invention belongs to the glycoside hydrolase 70 family according to the CAZy classification (www.cazy.org). CAZy stands for carbohydrate-active enzymes, a bioinformatics database for classifying enzymes active on sugars, i.e., capable of catalyzing their dissociation or their synthesis according to sequence or structure homologies, in particular their catalytic and carbohydrate binding modules. In the CAZy classification, the group of glycoside hydrolases (GH) consists of 173 families composed of enzymes active on sugars capable of catalyzing reactions of hydrolysis of glycosidic bonds or transglucosylation.

The α-transglucosylases of the GH70 family are typically produced naturally by lactic acid bacteria of the genera Streptococcus, Leuconostoc (abbreviated “L.”), Weisella, Oenococcus or Lactobacillus (abbreviated “Lb”).

The α-transglucosylases of the GH70 family according to the invention are active on sucrose, which means that the α-transglucosylases according to the invention specifically use sucrose or one of its analogs as glucosyl donor.

Advantageously, the sucrose analogs are compounds of formula (I):

X-α-D-glucopyranosyl (I)

wherein X is either a leaving group LG or an activated saccharide unit chosen in particular from O-β-D-galactopyranosyl-(1→4)-β-D-fructofuranosyl-(2→1) and α-L-sorbofuranoside.

According to the present invention, the LG group represents a chemical group that can be easily displaced by a nucleophile during a nucleophilic substitution reaction, the nucleophile being more particularly a group derived from the α-transglucosylase of the GH70 family.

Such a leaving group may more particularly be a halogen atom such as a chlorine, bromine or fluorine atom, a tosylate group (—OS(O)₂-(p-Me-C₆H₄)), or an optionally substituted aromatic group, especially an —O—C₆H₅—NO₂ group (preferably p-nitrophenyl or o-nitrophenyl), a 4-methylumbelliferyl group

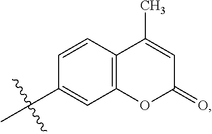 [SMILES: *c1ccc2c(C)cc(=O)oc2c1]

a resorufin group (phenoxazine 3-one group substituted in position 7) or a

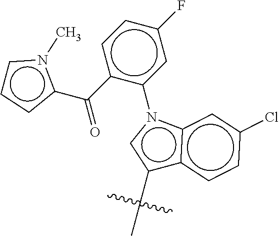

group (Aldol® 484).

For the purposes of the present invention, an aromatic group means an aryl or heteroaryl group optionally substituted, preferably by 1 to 2 substituents, especially chosen from a nitro group (—NO₂), a halogen, a C₁-C₄ alkyl group or an optionally substituted aryl.

For the purposes of the present invention, aryl means an aromatic hydrocarbon group, preferably comprising from 6 to 14 carbon atoms, and comprising one or more fused rings, for example a phenyl or naphthyl group. Advantageously, it is phenyl.

For the purposes of the present invention, heteroaryl or heteroaromatic means an aromatic group comprising 5 to 14 cyclic atoms, including one or more heteroatoms, advantageously 1 to 4 and even more advantageously 1 or 2, such as, for example, nitrogen or oxygen atoms, the other cyclic atoms being carbon atoms. Examples of heteroaryl groups are indyl, umbelliferyl or resorufin.

For the purposes of the present invention, halogen means fluorine, chlorine, bromine and iodine atoms. Preferably, it is a chlorine or fluorine atom.

For the purposes of the present invention, “C₁-C₄ alkyl” means a saturated, linear or branched monovalent hydrocarbon chain containing 1 to 4 carbon atoms, and preferably methyl.

An aryl is preferentially substituted by one or two substituents preferably chosen from a nitro group (—NO₂), a halogen, a C₁-C₄ alkyl group and/or a —C(O)-heteroaryl group, such as a 2-acyl-N-methylpyrrole group.

Thus, the sucrose analogues may especially be chosen from the group comprising α-D-glucopyranosyl fluoride, O-β-D-galactopyranosyl-(1→4)-β-D-fructofuranosyl-(2→1)-α-D-glucopyranoside (Lactulosucrose), p-nitrophenyl α-D-glucopyranoside, α-D-glucopyranosyl α-L-sorbofuranoside, 4-methylumbelliferyl α-D-glucosaminide (or 4-methylumbelliferyl 2-amino-2-deoxy-α-D-glucopyranoside, CAS No. [137687-00-4]), resorufin α-D-glucopyranoside (CAS No.: [136565-96-3]), Aldol® 484 alpha-D-glucopyranoside (CAS No: [2484872-56-0]) and mixtures thereof. The sucrose analogues may be, in particular, chosen from the group comprising α-D-glucopyranosyl fluoride, O-β-D-galactopyranosyl-(1→4)-β-D-fructofuranosyl-(2→1)-α-D-glucopyranoside, (Lactulosucrose), p-nitrophenyl α-D-glucopyranoside, α-D-glucopyranosyl α-L-sorbofuranoside and mixtures thereof.

Sucrose is preferred because this substrate is bio-based and inexpensive.

Advantageously, the GH70 family α-transglucosylase is a branching sucrase of the GH70 family, a glucansucrase of the GH70 family, or a mixture thereof.

In the present document, the terms “branching sucrase of the GH70 family”, sometimes abbreviated BRS, or “branching α-transglucosylase of the GH70 family” or “branching enzyme of the GH70 family” are used interchangeably and refer to an α-transglucosylase of the GH70 family capable of catalyzing the addition of glucosyl units into the main chain of a pre-existing dextran-type glucan forming branches. The nature of the branching bond (α-1,2; α-1,3; α-1,4 or α-1,6 bonds) and the length of the chains of glucosyl units constituting the branches vary according to the specificity of the branching sucrase considered.

Preferably, the branching sucrase of the GH70 family has as its amino acid sequence a sequence chosen from the group comprising GBD-CD2 ΔN123 (SEQ ID NO: 1), BRS-A (SEQ ID NO: 2), BRS-B Δ1 (SEQ ID NO: 3), BRS-C(SEQ ID NO: 4), BRS-D Δ1 (SEQ ID NO: 5), BRS-E Δ1 (SEQ ID NO: 6), or an amino acid sequence having at least 70%, 75%, 80%, 90%, 91%, 92%, 93%, 94%, 95%, 96%, 97%, 98%, 99% of sequence identity with at least one of SEQ ID NO: 1 to SEQ ID NO: 6.

The inventors have further shown that mutants of GBD-CD2 ΔN123 (SEQ ID NO: 1) were particularly efficient for glucosylation of hydroxy fatty acids. Preferably, the branching sucrase of the GH70 family is a mutant of GBD-CD2 ΔN123 (SEQ ID NO: 1) having an amino acid sequence chosen from GBD-CD2 ΔN123 W2135L F2136L (SEQ ID NO: 7), GBD-CD2 ΔN123 W2135L (SEQ ID NO: 8), GBD-CD2 ΔN123 W21351 F2136Y (SEQ ID NO: 9), GBD-CD2 ΔN123 W21351 F2136C (SEQ ID NO: 10), GBD-CD2 ΔN123 W2135V (SEQ ID NO: 11), or an amino acid sequence having at least 70%, 75%, 80%, 90%, 91%, 92%, 93%, 94%, 95%, 96%, 97%, 98%, 99% sequence identity with at least one of SEQ ID NO: 7 to SEQ ID NO: 11.

In the present document, the expression “glucansucrase of the GH70 family” sometimes abbreviated “GS” refers to a α-transglucosylase of the GH70 family capable of catalyzing the synthesis of α-glucans i.e., polysaccharides composed exclusively of glucosyl units linked to each other by a bonds. Glucansucrases include dextransucrases (sometimes abbreviated DSR), which synthesize dextrans, i.e., glucans whose main chain residues are bonded predominantly in α-1,6; reuteransucrases, which synthesize reuterans, i.e., glucans whose main chain residues are bonded at α-1,4 and α-1,6; mutansucrases, which synthesize mutans, i.e., glucans whose main chain residues are mainly bonded at α-1,3; alternansucrases, i.e., glucans whose main chain residues are bonded alternately at α-1,3 and α-1,6.

Preferably, the glucansucrase of the GH70 family has as amino acid sequence a sequence chosen from the group comprising DSR-OK (SEQ ID NO: 12), DSR-M DP (SEQ ID NO: 13), DRS-S vardelΔ4N (SEQ ID NO: 14), ASR Cdelbis-Δthio (SEQ ID NO: 15), GTF-SI (SEQ ID NO: 16), GTF-J (SEQ ID NO: 17), DSR-M Δ1 (SEQ ID NO: 18), or an amino acid sequence having at least 70%, 75%, 80%, 90%, 91%, 92%, 93%, 94%, 95%, 96%, 97%, 98%, 99% sequence identity with at least one of SEQ ID NO: 12 to SEQ ID NO: 18.

As used here, the term “sequence identity” or “identity” refers to the number (%) of pairings (identical amino acid residues) at the positions originating from an alignment of two polypeptide sequences. Sequence identity is determined by comparing sequences as they are aligned to maximize overlap and identity while minimizing sequence interruptions. In particular, the sequence identity can be determined using any one of the many global or local alignment algorithms, depending on the length of the two sequences. Sequences of similar lengths are preferably aligned using global alignment algorithms (e.g., Needleman & Wunsch, J. Mol. Biol 48:443, 1970) which optimally align the sequences over the entire length, while sequences of substantially different lengths are preferably aligned using a local alignment algorithm, for example the Smith and Waterman algorithm (Smith and Waterman, Adv. Appl. Math. 2:482, 1981) or the Altschul algorithm (Altschul et al. (1997) Nucleic Acids Res. 25:3389-3402; Altschul et al. (2005) FEBS J. 272:5101-5109). The alignment for the purpose of determining the amino acid sequence identity percentage can be carried out by any method known to the person skilled in the art, for example by using software available on websites such as http://blast.ncbi.nlm. nih.gov/or http://www.ebi.ac.uk/Tools/emboss/. The person skilled in the art can easily determine the appropriate parameters for measuring the alignment. For the purposes of the present invention, the amino acid sequence identity percentage values designate values generated using the “Protein Blast” (or Blastp) program in which the parameters are the default parameters (expect threshold: 10, word size: 6, matrix=BLOSUM62, gap costs: existence=11, extension=1, conditional compositional score matrix adjustment).

### Implementation of the Method

In one embodiment, step i) is carried out in solution at a controlled pH, in particular by the use of a buffer solution.

Advantageously, step i) is carried out at a pH value comprised between 5 and 8.

Step i) is advantageously carried out with an initial concentration of sucrose or of a sucrose analog comprised between 100 and 650 g·L⁻¹.

Step i) is preferably carried out with a molar ratio of sucrose or sucrose analog to hydroxy fatty acid of formula (II) comprised between 1 and 100, preferably between 10 and 100.

Preferably, step i) is carried out at a temperature comprised between 10° C. and 80° C.

Advantageously, step i) is carried out with a α-transglucosylase of the GH70 family in solution, in suspension or immobilized.

When a mixture of glycolipid is obtained at the end of step i), the method may also comprise a step ii) of purification of one or more glycolipid(s) of formula (I).

### Glycolipid

A second object according to the invention concerns a glycolipid of formula (I) as defined in the first object according to the invention.

The glycolipids according to the invention have the advantage of being biodegradable.

Advantageously, the glycolipid of the second object according to the invention is a glycolipid in which n is greater than or equal to 2, preferably is comprised between 2 and 7, more preferably is comprised between 2 and 4.

Advantageously, the glycolipid of the second object according to the invention is a glycolipid in which the fatty acid radical comprises at least 6, 7, 8, 9, 10 or 11 carbon atoms. Advantageously, the fatty acid radical comprises between 10 and 24 carbon atoms, preferably between 11 and 24 carbon atoms, preferably between 11 and 20 carbon atoms.

### First Variant: Glycolipid Comprising a Sulfanylated Carbon Chain

According to a first variant, the glycolipid comprises a sulfanylated carbon chain. In this embodiment, the glycolipid advantageously corresponds to formula (IA):

[Glc]ₙ-xOy-(CH₂)_{b}—S—(CH₂)ₐ—COOH
wherein
a is comprised between 9 and 14
b is greater than or equal to 2
the sum of a and b is less than 23.

Advantageously, the glycolipid of formula (IA) corresponds to the formula

[Glc]ₙ-xOy-(CH₂)₂—S—(CH₂)₁₀—COOH

### Second Variant: Glycolipid Comprising an Alkyl Carbon Chain

In a second variant according to the invention, the glycolipid of formula (I) comprises a carbon chain of alkyl type, advantageously of linear alkyl type.

In this embodiment, the glycolipid of formula (I) advantageously corresponds to formula (IB):

[Glc]ₙ-xOy-(CH₂)ₐ—COOH (IB)

wherein a is comprised between 3 and 23, preferably between 10 and 15.

Advantageously, the glycolipid of formula (IB) corresponds to the formula [Glc]ₙ-xOy-(CH₂)ₐ—COOH.

### Third Variant: Glycolipid Comprising a Hydroxylated Carbon Chain

In a third variant according to the invention, the fatty acid radical is substituted by one or two hydroxyl groups.

In this embodiment, the glycolipid of formula (I) is preferably a glycolipid in which:
The Cy carbon atom is a carbon atom positioned at the omega end, and the carbon chain of the fatty acid radical is substituted by one or two hydroxyl groups positioned along the carbon chain, or
The Cy carbon atom is a carbon atom positioned along the carbon chain, and the carbon chain of the fatty acid radical is substituted by two hydroxyl groups, with one hydroxyl group positioned along the carbon chain and one hydroxyl group positioned in the omega position of the carbon chain, or with two hydroxyl groups positioned along the carbon chain.

### Use of the Glycolipid as an Antimicrobial Agent

The inventors have discovered that the glycolipid according to the invention has antimicrobial properties, in particular antibacterial properties.

An object of the invention is therefore also the use of the glycolipid as defined above as an antimicrobial agent, especially as an antibacterial agent.

For the purposes of the invention, the term “antimicrobial agent” designates a compound capable of destroying microbes (i.e., a microbiocide) or slowing their growth (i.e. a microbiostatic agent). Microbe means unicellular or pluricellular viruses or microorganisms.

By way of example, the glycolipid according to the invention can be used as a preservative which inhibits the development of microorganisms and makes it possible to increase the shelf life of products, in particular in the cosmetic, pharmaceutical or food fields.

### Use of the Glycolipid as a Surfactant

The inventors have also discovered that the glycolipid according to the invention can also be used as a surfactant, in particular as an emulsifying agent.

An object of the invention is therefore also the use of the glycolipid as defined above as a surfactant, especially as an emulsifying agent.

For the purposes of the invention, a surfactant is a substance modifying the surface tension between two surfaces; an emulsifying agent is a surfactant which facilitates the formation of an emulsion or improves its stability by reducing its rate of aggregation and/or coalescence.

In particular, the glycolipid according to the invention can be used as an emulsifier for the preparation of an oil-in-water emulsion.

For the purposes of the invention, an oil-in-water emulsion comprises an oil phase dispersed within an aqueous phase, and a water-in-oil emulsion comprises an aqueous phase dispersed within an oil phase.

The inventors have discovered that, surprisingly, when a glycolipid according to the invention is used as emulsifying agent for the preparation of an oil-in-water emulsion, this emulsion is sensitive to pH, more particularly since the latter is kinetically stable as long as the pH of the aqueous phase of the emulsion is greater than a threshold potential of hydrogen pHs, and that it destabilizes when this pH is adjusted so as to be less than pHs.

### Ph Sensitive Emulsion

Another object according to the invention is therefore an oil-in-water emulsion comprising an oil phase dispersed in an aqueous phase and at least one glycolipid of formula (I), characterized in that the emulsion is stable when the pH of the aqueous phase is greater than a threshold potential of hydrogen pHs.

In the present invention, the expression “kinetically stable as long as the pH of the aqueous phase of the emulsion is greater than a threshold potential of hydrogen pHₛ” means that the pH of the aqueous phase of the emulsion must be greater than pHₛ for the emulsion to be stable.

Advantageously, the pHₛ corresponds to the pKa of the glycolipid according to the invention used as emulsifier. In the case where the emulsion comprises a mixture of glycolipids according to the invention, the pHₛ advantageously corresponds to the pKa of the mixture of glycolipids according to the invention.

Preferably, the threshold hydrogen potential pHₛ ranges from 2 to 5, and preferably from 2 to 4, and more preferably is approximately 4.

When the pH of the aqueous phase of the emulsion is lower than the pHₛ as described above, the emulsion destabilizes in a few minutes, typically from 5 to 20 minutes, and is completely destroyed in a few hours, typically from 2 to 24 hours.

In one embodiment, the emulsion comprises a mixture of glycolipids according to the invention as described above.

Without wishing to be bound by a particular theory, the inventors believe that the instability of emulsions observed for low pH values reflects the existence of a strong electrostatic component in the stabilization of emulsions by glycolipids according to the invention.

### Nature and Quantity of the Oil and Aqueous Phases

Preferably, the aqueous phase advantageously represents at least 50% by weight of the emulsion, relative to the total weight of the emulsion. Preferably, the emulsion according to the invention comprises at least 50%, 55%, 60%, 65%, 70%, 75%, 80% or 85% by weight of aqueous phase, relative to the total weight of the emulsion.

The quantity of aqueous phase in the emulsion according to the invention is especially comprised between 50 and 80% by weight, preferably between 50 and 70% by weight and more preferentially between 50 and 60% by weight relative to the total weight of the emulsion.

Advantageously, the aqueous phase represents between 50 and 70% by weight of the emulsion.

The aqueous phase of the emulsion according to the invention mainly comprises water, a glycolipid or a mixture of glycolipids according to the invention and optionally one or more water-miscible compounds.

Preferably, the aqueous phase comprises, in % by weight of aqueous phase, from 0.5 to 5%, preferably from 0.5 to 3%, more preferably from 1 to 3% of a glycolipid or of a mixture of glycolipids according to the invention.

The aqueous phase may also comprise ionic species, pH regulators, active ingredients, preservatives or alternatively dyes, said active ingredients, preservatives and dyes being water-soluble or water-dispersible.

The oil phase of the emulsion according to the invention is a fatty phase comprising at least one fatty substance chosen from fatty substances which are liquid at room temperature (20-25° C.) or oils, volatile or otherwise, of vegetable, mineral or synthetic origin, and mixtures thereof. The term “oil” means a fatty substance that is liquid at room temperature (25° C.). The oil phase may also comprise any liposoluble or lipodispersible additive.

For an application in the cosmetic or pharmaceutical fields, these oils are chosen from physiologically acceptable oils.

Oils that can be used in the emulsion according to the invention include, for example, hydrocarbon oils of animal origin; hydrocarbon oils of vegetable origin; synthetic esters and ethers, especially fatty acids; linear or branched hydrocarbons, of mineral or synthetic origin, such as paraffin oils, volatile or otherwise, and derivatives thereof; partially hydrocarbon-based and/or silicone-based fluorinated oils; silicone oils; and mixtures thereof.

Among the hydrocarbons of molecular formula CₙHₘ with n and m being two integers, linear, aromatic and cyclic hydrocarbons can be used, regardless of their boiling point. More particularly, hydrocarbons that may be present in the oil phase according to the invention include cyclopentane (C₅H₁₀), hexane (C₆H₁₄), methylcyclohexane (C₇H₁₄), heptane (C₇H₁₆), decane (C₁₀H₂₂), dodecane (C₁₂H₂₆), hexadecane (C₁₆H₃₄), and toluene (C₇H₈).

The oil phase in the emulsion according to the invention may be composed of a single oil, in particular a single hydrocarbon, or may also be composed of a mixture of two, three or even four different oils.

In the emulsion, the oil phase is typically in the form of drops of oil phase suspended in the aqueous phase.

The drops of oil phase are preferably monodisperse.

The particle size distribution of the oil phase drops can be characterized in terms of the mean volume drop diameter, D (4.3), and the polydispersity in size, P, defined by:

$$D ( 4 , 3 ) = \frac{\sum {N}_{i} {D}_{i}^{4}}{\sum {N}_{i} {D}_{i}^{3}} \qquad [ Chem 1 ]$$

$$and$$

$$P = \frac{1}{{D}_{m}} \frac{\sum {N}_{i} {D}_{i}^{3} ( {D}_{m} - D )}{\sum {N}_{i} {D}_{i}^{3}} \qquad [ Chem 2 ]$$

where N_{I} is the number of drops of diameter Dᵢ, and Dₘ is the median diameter (diameter which divides the distribution into two parts of equal areas).

The median diameter of the drops can be measured from a size histogram obtained by measuring the individual diameters of an assembly of drops (minimum 500) on one or more images taken by light microscopy or by measuring static light scattering.

The drops of the oil phase advantageously have a volume mean diameter of around a few micrometers. Thus, the droplets generally have a volume mean diameter comprised between 2 and 10 μm, preferably between 3 and 6 μm, more preferably between 4 and 5 μm.

### Dry Emulsion

The inventors have also shown that the emulsion according to the invention can be subjected to a treatment in order to produce a dry emulsion, the treatment consisting of eliminating most or all of the aqueous phase of the emulsion according to the invention by drying or lyophilization.

An object according to the invention therefore concerns an emulsion according to the invention in the form of a dry emulsion.

For the purposes of the invention, a dry emulsion is in the form of a powder consisting of oily particles capable of restoring an oil-in-water emulsion after rehydration, i.e., after dispersion of the dry emulsion in an aqueous phase. The expressions “emulsion in the form of a dry emulsion” and “dry emulsion” are used interchangeably.

Preferably, the dry emulsion comprises drops of oil phase, a glycolipid according to the invention and less than 20%, preferably less than 10% by weight of aqueous phase, relative to the total weight of the dry emulsion. Advantageously, the dry emulsion comprises at most 1% or 2% by weight of aqueous phase, relative to the total weight of the dry emulsion. Also advantageously, the dry emulsion is devoid of aqueous phase.

The oil phase drops have a mean size of around 4 μm.

According to a preferred embodiment, the dry emulsions according to the invention contain a protecting saccharide compound, especially chosen from the group consisting of sucrose, lactose, fructose, trehalose, dextrins, maltodextrins, yellow dextrins, invert sugars, sorbitol, polydextrose, starch syrup, glucose syrup and mixtures thereof. Preferably, the protecting saccharide compound is lactose.

The dry emulsion is advantageously obtained by freezing an emulsion according to the invention, typically at a temperature comprised between −50° C. and −100° C. and by lyophilizing it.

### Method for Preparing an Emulsion

The invention also concerns the preparation of an emulsion according to the invention, comprising:
a) preparing an aqueous phase and an oil phase, wherein the oil phase and/or the aqueous phase comprises the glycolipid according to the invention;
b) combining the aqueous phase and the oil phase and stirring until an emulsion is obtained.

Preferably, the oil phase and/or the aqueous phase comprise the glycolipid according to the invention in an aqueous phase/glycolipid mass ratio comprised between 60 and 10.

Preferably, the aqueous phase of step a) comprises, in % by weight of aqueous phase, from 0.5 to 5%, preferably from 0.5 to 3%, more preferably from 1 to 3% of glycolipid according to the invention. In one embodiment, the oil phase does not comprise glycolipid according to the invention.

During step a), the pH of the aqueous phase is preferably adjusted so as to be greater than the pKa of the glycolipid according to the invention.

The oil phase and the aqueous phase are combined in the most appropriate order, as is known to the person skilled in the art. In a particular embodiment, step b) is carried out by adding the oil phase to the aqueous phase while stirring the combination obtained.

### Use of the Emulsion for Cleaning Surfaces

Another object according to the invention concerns the use of an emulsion according to the invention for cleaning surfaces.

Indeed, surprisingly, the inventors have discovered that when a drop of emulsion according to the invention is deposited on a solid substrate, such as a glass slide, or a plastic support, the latter moves, spreads and contracts in a spasmodic and quasi-periodic manner on the support. This makes the emulsion self-spreadable or even self-stirred, which is of interest in the field of surface cleaning. Without wishing to be bound by a particular theory, the inventors attribute these properties to convective phenomena taking place within the drop of emulsion.

### EXAMPLES

### Enzymes Studied, Organisms of Origin and Specificity

The enzymes used in the examples are listed in Table 1.

TABLE 1
GH70 α-transglucosylases, binding specificity
during synthesis of the natural polymer.

| | | Reaction specificity | | | | |
|---|---|---|---|---|---|---|
| SEQ ID NO: | Name | B/P | α-1,2 | α-1,3 | α-1,6 | α-1,4 |
| 1 | GBD-CD2 ΔN123 | B | 100% | — | — | — |
| 2 | BRS-A | B | 100% | — | — | — |
| 3 | BRS-B Δ1 | B | — | 100% | — | — |
| 4 | BRS-C | B | — | 100% | — | — |
| 5 | BRS-D Δ1 | B | 100% | — | — | — |
| 6 | BRS-E Δ1 | B | 66% | — | 33% | — |
| 12 | DSR-OK | P | 2% | — | 98% | — |
| 13 | DSR-DP | P | nd | nd | nd | nd |
| 14 | DSR-S | P | — | 4% | 96% | — |
| | vardel Δ4N | | | | | |
| 15 | ASR | P | | 55% | 45% | |
| | Cdelbis-Δthio | | | | | |
| 16 | GTF-SI | P | | 100% | | |
| 17 | GTF-J | P | | 95% | 5% | |
| 18 | DSR-M Δ1 | P | — | — | 100% | — |

P: polymerase, i.e., glucansucrase GH70;
B: GH70 branching sucrase;
nd = not determined

The origin of the enzymes in Table 1 are listed in Table 2.

TABLE 2
Organisms of origin of GH70 α-transglucosylases

| SEQ ID NO: | Name | Organism of origin |
|---|---|---|
| 1 | GBD-CD2 ΔN123 | Leuconostoc citreum NRRL B-1299 |
| 2 | BRS-A | Leuconostoc citreum NRRL B-1299 |
| 3 | BRS-B Δ1 | Leuconostoc citreum NRRL B-742 |
| 4 | BRS-C | Leuconostoc fallax KCTC 3537 |
| 5 | BRS-D Δ1 | Lactobacillus kunkei EFB6 |
| 6 | BRS-E Δ1 | Leuconostoc mesenteroides |
| | | KFRI-MG |
| 12 | DSR-OK | Oenococcus kitaharae DSM 17330 |
| 13 | DSR-DP | Leuconostoc citreum NRRL B-1299 |
| 14 | DSR-S vardel Δ4N | Leuconostoc mesenteroides |
| | | NRRL B-512F |
| 15 | ASR Cdelbis-Δthio | Leuconostoc mesenteroides |
| | | NRRL B-1355 |
| 16 | GTF-SI | Streptococcus mutans |
| 17 | GTF-J | Streptococcus salivarius |
| 18 | DSR-M Δ1 | Leuconostoc citreum NRRL B-1299 |

Heterologous Expression of Enzymes in Escherichia coli

The genes coding for the enzymes mentioned above are cloned into vectors allowing recombinant expression in E. coli (see Table 3).

Recombinant enzymes are produced from E. coli cells BL21 Star DE3 (SEQ ID NO: 18, 13, 1, 3, 4, 5, 6, 16, and 17), BL21 AI DE3 (SEQ ID NO: 12, 14, and 2) or Top 10 (SEQ ID NO: 15) transformed with the plasmid containing the gene of the targeted enzymes (see Table 3).

TABLE 3
Plasmids of the enzymes used and expression
systems adapted to the different enzymes.

| | | | Conditions of modified | | | |
|---|---|---|---|---|---|---|
| SEQ ID | | | medium ZYM5052 | | | |
| NO: | Enzyme | Plasmid | Gly | Glu | Inductor | Activity buffer |
| 1 | GBD-CD2 | pET53 | 1.5% | 0.05% | 1% α-Lac | 50 mM acetate |
| | ΔN123 | | | | | pH = 5.75 |
| 2 | BRS-A | pBAD49 | 0.5% | 0.05% | 0.01% | 50 mM acetate |
| | | | | | L-Ara | pH = 5.75 |
| 3 | BRS-B Δ1 | pET55 | 1% | 0% | 0.1% α-Lac | 50 mM acetate |
| | | | | | | pH = 5.75 |
| 4 | BRS-C | pET55 | 1% | 0% | 0.1% α-Lac | 50 mM acetate |
| | | | | | | pH = 5.75 |
| 5 | BRS-D Δ 1 | pET53 | 1% | 0% | 1% α-Lac | 50 mM acetate |
| | | | | | | pH = 5.75 |
| 6 | BRS-E Δ 1 | pET60 | 1% | 0% | 0.1% α-Lac | 50 mM acetate |
| | | | | | | pH = 5.75 |
| 12 | DSR-OK | pET53 | 1% | 0% | 1% α-Lac | 50 mM acetate |
| | | | | | | pH = 5.75 |
| 13 | DSR-DP | pET55 | 1% | 0% | 0.1% α-Lac | 50 mM acetate |
| | | | | | | pH = 5.75 |
| 14 | DSR-S | pBAD49 | 0.5% | 0.05% | 0.01% | 50 mM acetate |
| | vardel | | | | L-Ara | pH = 5.75 |
| | Δ 4N | | | | | |
| 15 | ASR | pBAD49 | 0.5% | 0.05% | 0.01% L- | 50 mM acetate |
| | Cdelbis- | | | | Ara | pH = 5.2 |
| | Δthio | | | | | |
| 16 | GTF-SI | pET21a | 1% | 0% | 1% α-Lac | 10 mM |
| | | (+) | | | | phosphate |
| | | | | | | pH = 6.5 |
| 17 | GTF-J | pET21a | 1% | 0% | 1% α-Lac | 10 mM |
| | | (+) | | | | phosphate |
| | | | | | | pH = 6.5 |
| 18 | DSR-M Δ1 | pET55 | 1% | 0% | 0.1% α-Lac | 50 mM acetate |
| | | | | | | pH = 5.75 |

(Gly: Glycerol; Glu: Glucose; α-Lac: α-lactose; L-Ara: L-arabinose)

Three hundred microliters of the transformation mixture inoculated a volume of 30 ml of LB (lysogeny broth) supplemented with 100 μg·mL⁻¹ of ampicillin. The medium is incubated overnight at 37° C. in order to prepare a preculture.

1-L cultures in modified ZYM5052 medium (Studier, 2005) whose characteristics are presented in Table 3 are inoculated at an initial OD_{λ=600 nm} of 0.05 from the preculture of the previous day, then incubated for 26 hours at 21° C. and 150 rpm.

At the end of fermentation, the culture media are centrifuged (15 min, 6500 rpm, 4° C.) and the pellets are concentrated at an OD_{λ=600 nm} of 80 in activity buffer (see Table 3). The cells are then ultrasonically broken according to the following protocol: 5 cycles of 20 seconds at 30% of the maximum power of the probe, cold, with a 4-minute rest interval in ice. The sonication supernatants containing the soluble enzymes of interest are then recovered after 30 minutes of centrifugation (10,000 rpm, 10° C.) and stored at 4° C.

### Determining Enzyme Activity by Assay of Reducing Sugars in DNS.

Enzyme activity is determined by measuring the initial rate of production of reducing sugars using the 3,5-dinitrosalicylic acid (DNS) method (Miller, 1959).

One enzyme unit represents the amount of enzyme that releases one μmole of fructose per minute, at 30° C., from 100 g·L⁻¹ sucrose in 50 mM sodium acetate buffer, pH 5.75. The activity is determined by measuring the initial rate of production of reducing sugars using the 3,5-dinitrosalicylic acid (DNS) method.

During kinetics, 100 μL of reaction medium are removed and the reaction is stopped by adding an equivalent volume of DNS. The samples are then heated for 5 min at 95° C., cooled in ice, diluted to half in water, and the absorbance is read at 540 nm. A standard range of 0 to 2 g·L⁻¹ of fructose establishes the link between absorbance value and reducing sugar concentration.

### Example 1: Screening Enzymes in Acceptor Reaction with Different Hydroxy Fatty Acids

### 1.1. Materials and Methods

### Reaction Conditions

Acceptor reactions are carried out in conical microtubes, in a volume of 1 mL. Screening for GH70 family enzymes was performed under the following conditions:
[sucrose]₀=292 mM (100 g·L⁻¹)
[hydroxy fatty acid]₀=10 mM,
10% v/v of DMSO
50 mM sodium acetate buffer at pH=5.75
enzymatic activity: 1 U·mL⁻¹
temperature: 30° C.
stirring: 800 rpm

The hydroxy fatty acid is initially dissolved in 100% DMSO.

The hydroxy fatty acids (hereinafter HFAs) tested are respectively:
a sulfanylated hydroxy fatty acid: 11-[2-[hydroxyalkyl]-sulfanyl]-undecanoic acid (hereinafter HESUA)
a ω-hydroxy fatty acid: 11-hydroxyundecanoic acid (hereinafter HUDA)
a poly-hydroxy fatty acid: erythro-aleuritic acid (hereinafter EAA)

The final concentration of DMSO in the reaction medium is 10% (v/v). The reaction is initiated by the addition of a volume of cell lysate sufficient to obtain an enzymatic activity in reaction of 1 U·mL⁻¹. The reactions are incubated at 30° C. and stirred at 800 rpm using an Eppendorf ThermoMixer C. After 24 h, the enzymes were denatured at 95° C. for 5 min.

### Analytical Techniques

For HPLC analysis, the reaction media are diluted to half in absolute ethanol. This dilution makes it possible to remove potential high molecular weight polymer by precipitation.

The separation of the lipid acceptors and their glucosylated forms is carried out in reverse phase with a Synergi™ Fusion-RP column (porosity of 80 Å, particle size of 4 μm, C18 grafting with polar termination, Phenomenex, USA). This column is maintained at 30° C. on a Thermo U3000 HPLC system coupled to a Corona CAD Véo (charged aerosol detector) (Thermo Fisher Scientific, USA). The nebulization temperature is set at 50° C. and the filter is maintained at 3.2 seconds.

The mobile phase is composed of a mixture of ultrapure water (solvent A) and HPLC grade acetonitrile (solvent B) each containing 0.05% (v/v) formic acid. Elution is carried out at a flow rate of 1 mL·min⁻¹ according to the following gradient:
Between 0 min and 5 min, a first phase of elution at 0% (v/v) of channel B allows the elimination of residual simple sugars (residual fructose, glucose, leucrose, sucrose or possible short oligosaccharides),
A second isocratic elution phase at a percentage of 35% of channel B is carried out for 25 minutes to separate the different glucosylated lipid compounds,
A final phase of 10 minutes at 75% of channel B allows regeneration of the column.

### Acceptor Conversion Rate

The ability of enzymes to glucosylate an acceptor, here HESUA, HUDA or EAA, is determined by measuring the conversion rate of the acceptor between the beginning and the end of the reaction. The conversion rate is calculated as follows:

$${X}_{acceptor} ( \% ) = ( 1 - \frac{{[ Acceptor ]}_{f}}{{[ Acceptor ]}_{0}} ) \times 100 \qquad [ Math . 1 ]$$

where X_{acceptor} is the conversion rate of the acceptor, [Acceptor] o its initial concentration, and [Acceptor] f its final concentration.

### 1.2. Results

### Glucosylation of HESUA

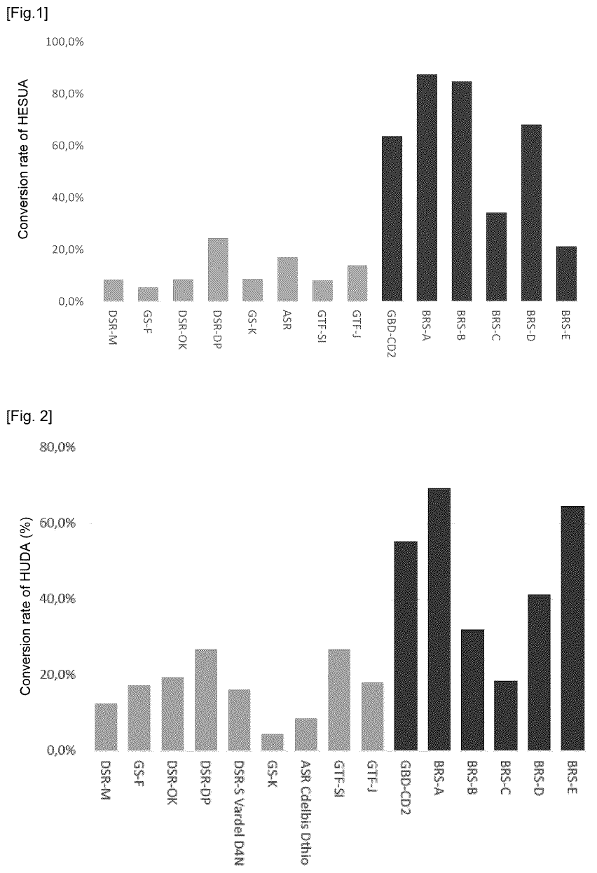

FIG. 1 illustrates the ability of enzymes to glucosylate HESUA under the conditions outlined above.

The enzymes of the GH70 family tested were found to be active on HESUA.

The branching enzymes BRS-A, BRS-B Δ1, BRS-B, BRS-E Δ1 and GBD-CD2 ΔN123 proved to be particularly active. Indeed, conversion rates ranging from 20% to greater than 80% under the screening conditions tested were obtained with these enzymes.

### Glucosylation of HUDA

FIG. 2 illustrates the ability of enzymes to glucosylate HUDA under the conditions outlined above.

All the enzymes of the GH70 family tested were found to be active on HUDA.

The branching enzymes BRS-A, BRS-B Δ1, BRS-B, BRS-E Δ1 and GBD-CD2 ΔN123 proved to be particularly active. Indeed, conversion rates ranging from 18% to greater than 60% under the screening conditions tested were obtained with these enzymes.

### Glucosylation of EAA

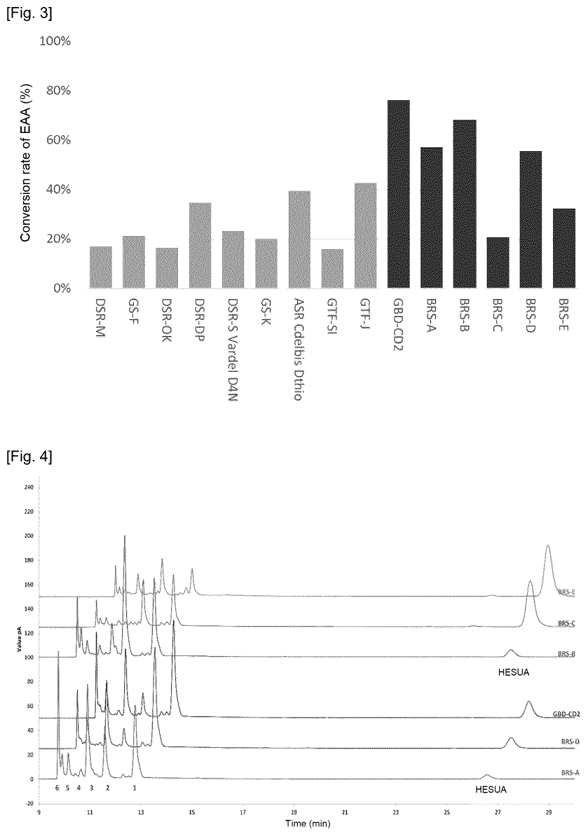

FIG. 3 illustrates the ability of enzymes to glucosylate EAA under the conditions outlined above.

All the enzymes of the GH70 family tested were found to be active on EAA.

The branching enzymes BRS-A, BRS-B Δ1, BRS-B, BRS-E Δ1 and GBD-CD2 ΔN123 proved to be particularly active. Indeed, conversion rates ranging from 20% to greater than 60% under the screening conditions tested were obtained with these enzymes.

### Example 2: Comparison of Glucosylation Product Profiles of HESUA, HUDA and EAA by Branching Enzymes BRS-A, BRS-B Δ1, BRS-C, BRS-D Δ1, BRS-E Δ1 and GBD-CD2 ΔN123

### 2.1. Materials and Methods

The reactions are carried out at 30° C. under 800 rpm stirring in the presence of 438.5 mM sucrose, 10 mM GHA (HESUA, HUDA or EAA), 10% v/v of DMSO and a 50 mM sodium acetate buffer at pH=5.75 and BRS-A; BRS-B Δ1; BRS-C; BRS-D Δ1, BRS-E 41 or GBD-CD2 ΔN123 (enzymatic activity of 1 U. mL⁻¹).

### 2.2. Results

### 2.2.1. Profiles of Glucosylation Products of HESUA

FIG. 4 illustrates the diversity of glucosylation products obtained under the conditions outlined above.

Glucosylation products are eluted between 10 and 16 minutes from HPLC analysis. The product detected at 26.5 minutes corresponds to the residual HESUA.

For each enzyme tested, the chromatographic profiles exhibit numerous glucosylation peaks. For example, six major products are obtained when BRS-A catalyzes the reaction.

The profile of glucosylation products is dependent on the enzyme considered.

The majority of glucosylation products appear to be more numerous with the branching enzymes BRS-A, BRS-D Δ1 and GBD-CD2 ΔN123 specific for the α-1,2 bond, especially in the products eluted in the early stages of the analysis. Indeed, three to four high intensity peaks are observable with these enzymes.

On the contrary, only two main products are obtained with BRS-B Δ1, BRS-C and BRS-EΔ1.

With the exception of enzymes BRS-C and BRS-E Δ1, glucosylation efficiency is very high with HESUA as acceptor, particularly for BRS-A, BRS-B Δ1, BRS-D Δ1, and GBD-CD2 ΔN123. Indeed, the conversion rates of HESUA obtained with 438.5 mM (150 g·L⁻¹) of sucrose are 94%, 84% and 75% for BRS-A, BRS-D Δ1, and GBD-CD2 ΔN123 respectively (BRS specific for the α-1,2 bond) and 89%, 26%, 22% for BRS-B Δ1, BRS-C and BRS-E 41 (BRS specific for the α-1,3 bond).

### 2.2.2. Profiles of Glucosylation Products of HUDA

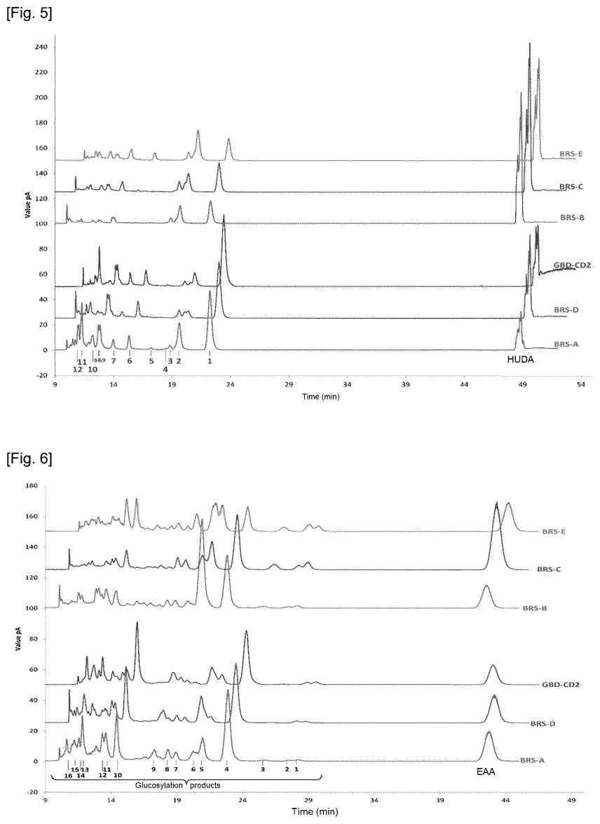

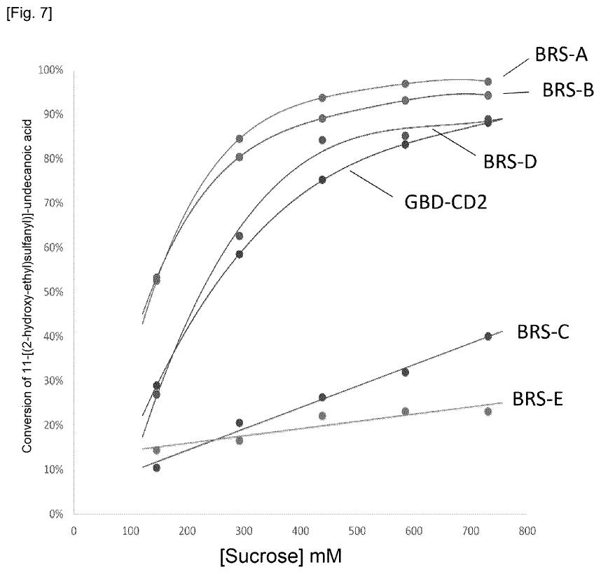

FIG. 5 illustrates the diversity of glucosylation products obtained under the conditions outlined above.

The glucosylation products obtained are eluted between 10 and 25 minutes. The product detected at 49 minutes corresponds to residual 11-hydroxyundecanoic acid (HUDA). The product profiles obtained are complex with numerous glucosylation peaks detected (a dozen different products for BRS-A for example).

The profile of glucosylation products is also dependent on the enzyme considered. The major products 1 and 2 are, for example, in variable proportions according to the enzymes: a high production of glucosylation product 1 is observed with GBD-CD2 ΔN123; on the contrary, a production of the same order of magnitude of the two glucosylation products (1 and 2) is obtained with BRS-E Δ1.

The products eluted in the first moments of the analysis (between 10 and 15 minutes) appear in higher quantities in reactions catalyzed by enzymes specific to the α-1,2 bond, i.e., BRS-A, BRS-D Δ1 and GBD-CD2 ΔN123.

Glucosylation efficiency is also dependent on the enzyme considered.

Indeed, the conversion rates of HUDA obtained are 78%, 40%, 17%, 54%, 32% and 67% for BRS-A, BRS-B Δ1, BRS-C, BRS-D Δ1, BRS-E Δ1 and GBD-CD2 ΔN123 respectively under the conditions illustrated in FIG. 5.

### 2.2.3. Profiles of Glucosylation Products of EAA

FIG. 6 illustrates the diversity of glucosylation products obtained under the conditions outlined above.

The glucosylation products obtained are eluted between 10 and 30 minutes of the HPLC analysis, the product detected at 43 minutes corresponding to the residual EAA. The glucosylation product profiles appear complex, since numerous glucosylation peaks are detected (more than 16 major products for BRS-A for example).

The profile of glucosylation products is very dependent on the enzyme considered. Indeed, while peak 4 is common to all the enzymes, peak 5 is characteristic of the enzyme BRS-B Δ1. Peak 10 is essentially present in the products obtained with the enzymes BRS-A and BRS-D Δ1 two enzymes specific for the α-1,2 bond. Glucosylation products 1 to 3 are essentially produced by BRS-B Δ1 and BRS-E Δ1, two enzymes specific for the α-1,3 bond.

Glucosylation efficiency is also dependent on the enzyme considered. Indeed, the conversion rates of EAA obtained are 63%, 74%, 30%, 60%, 65% and 74% for BRS-A, BRS-B Δ1, BRS-C, BRS-D Δ1, BRS-E Δ1 and GBD-CD2 ΔN123 respectively under the conditions illustrated in FIG. 6.

### Example 3: Effect of the Initial Concentration of Sucrose on the Profile of Glucosylation Products of HESUA, HUDA and EAA Obtained with BRS-A; BRS-B Δ1; BRS-C; BRS-D Δ1, BRS-E Δ1 or GBD-CD2 ΔN123

### 3.1. Materials and Methods

### Profile of Glucosylation Products

The reactions are carried out at 30° C. with stirring at 800 rpm in the presence of 146 mM, 292 mM, 439 mM, 585 mM or 731 mM sucrose; 10 mM GHA (HESUA, HUDA or EAA); 10% v/v of DMSO and a 50 mM sodium acetate buffer at pH=5.75 in the presence of BRS-A; BRS-B Δ1; BRS-C; BRS-D Δ1, BRS-E Δ1 or GBD-CD2 ΔN123 (enzymatic activity 1 U·mL⁻¹, T=30° C.).

### 3.2. Results

### 3.2.1. Effect of Initial Sucrose Concentration on Conversion Rate and Profile of HESUA Glucosylation Products

Increasing the initial sucrose concentration optimizes the conversion rate of HESUA (FIG. 7). This effect is particularly marked for the enzymes BRS-A, BRS-B Δ1, BRS-D Δ1 and GBD-CD2 ΔN123. Indeed, the conversion rate between 25% and 55%, obtained at an initial concentration of 146 mM, increases to a rate greater than 85% (up to 98% for BRS-A).

The increasing initial sucrose concentrations also have an effect on the profiles of the glucosylation products (not shown). For example, for the four enzymes BRS-A, BRS-B Δ1, BRS-D Δ1 and GBD-CD2 ΔN123, the glucosylation products eluted in the first moments of the analysis (certainly products with a higher degree of glucosylation) are clearly favored by the increase in the initial sucrose concentration.

### 3.2.2. Effect of Initial Sucrose Concentration on Conversion Rate and Profile of Glucosylation Products of HUDA

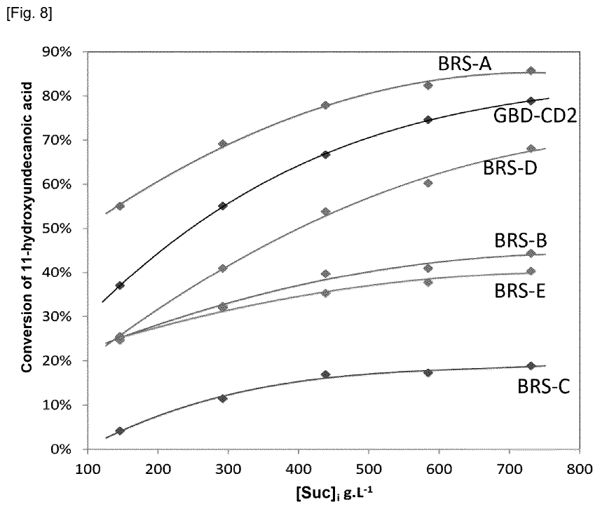

Increasing the initial sucrose concentration optimizes the conversion rate of HUDA (FIG. 8).

This effect is especially marked for enzymes with α-1,2 specificity. Indeed, the conversion rate comprised between 25% and 55%, obtained at an initial concentration of 146 mM with BRS-A, BRS-D Δ1 and GBD-CD2 ΔN123 increases to a rate comprised between 60% and 85% for a concentration greater than 146 mM. This effect of the initial concentration of sucrose is less marked for BSR-B Δ1, BSR-C and BSR-E Δ1.

In comparison, the degree of conversion obtained with enzymes with α-1,3 specificity does not exceed 40% for an initial sucrose concentration of 730 mM.

The increasing initial sucrose concentrations also have an effect on the overall profile of the glucosylation products (not shown). For example, for the three branching enzymes BRS-A, BRS-D Δ1 and GBD-CD2 ΔN123, the glucosylation products eluted in the first moments of the analysis are clearly favored by the increase in the initial concentration of sucrose. These products are certainly products with a higher degree of glucosylation. In the case of α-1,3 branching enzymes, this effect is much less marked.

### 3.2.3. Effect of Initial Sucrose Concentration on Conversion Rate and Profile of Glucosylation Products of EAA

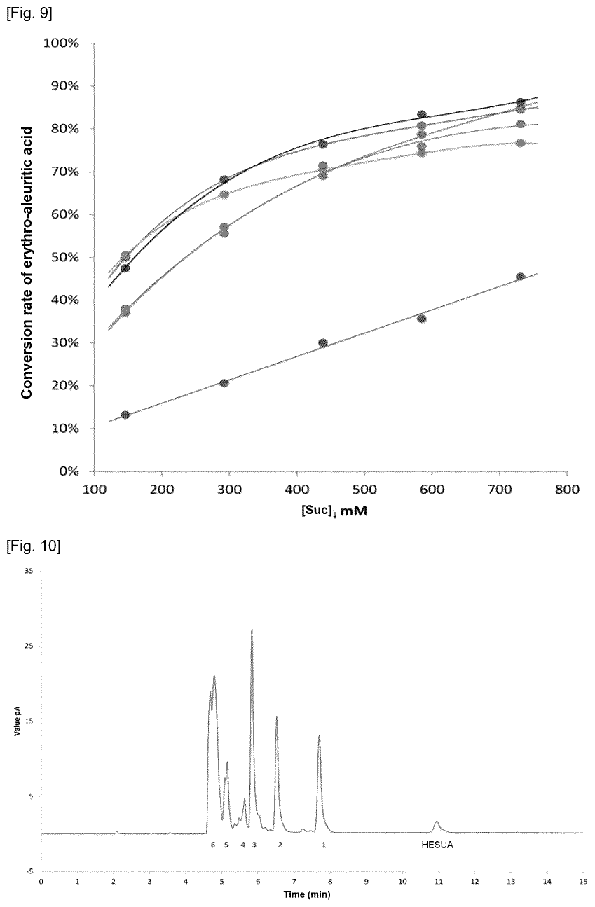

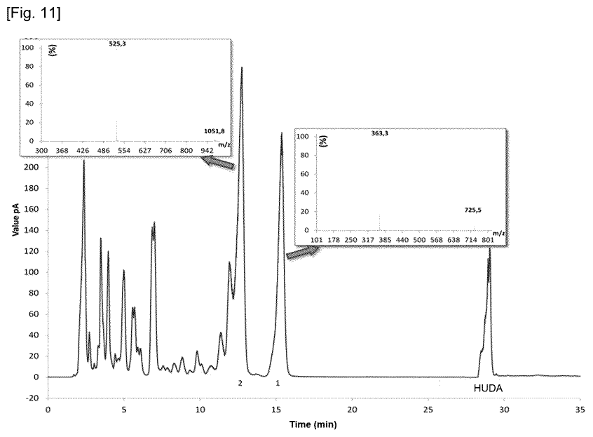

Increasing the initial sucrose concentration decreases the peak residual EAA. The conversion of the acceptor is therefore optimized. Indeed, the conversion rate comprised between 37 and 50% obtained at an initial concentration of 146 mM increases to a rate comprised between 76% and 85% depending on the enzymes considered (BRS-B Δ1, BRS-D Δ1, BRS-E Δ1 and GBD-CD2 ΔN123). This effect of initial sucrose concentration is less marked for the enzyme BRS-C. This optimization of EAA conversion is illustrated in FIG. 9.

The increasing initial sucrose concentrations also have an effect on the overall profile of the glucosylation products (not shown). For example, for the two branching enzymes BRS-A and BRS-D Δ1 (which glucosylate dextran at α-1,2), the glucosylation products eluted in the first moments of the analysis are clearly favored with the increase in the initial concentration of sucrose. These products are certainly products with a higher degree of glucosylation.

In the case of α-1,3 branching enzymes, the synthesis of glucosylation product 4 is disadvantaged in favor of that of other glucosylation products, especially glucosylation product 5 in the case of the BRS-B enzyme.

### Example 4: Structural Characterization of the Main Glucosylation Products of HESUA by BRS-A, of HUDA by BSR-B Δ1 and of EAA by BSR-B Δ1

### 4.1. Production and Structural Characterization of HESUA Glucosylation Products

### 4.1.1. Production of a Batch of HESUA Glycolipids at Gram Scale

Glucosylation of HESUA is catalyzed by the enzyme BRS-A on 500 mg of HESUA. The reaction conditions are as follows:
[sucrose]₀=438.5 mM
[HESUA]₀=10 mM (initially dissolved in DMSO at 100 mM)
sodium acetate buffer at 50 mM and at pH=5.75
ultrapure water qs 200 mL
activity of BRS-A in the reaction: 1 U·mL⁻¹

The reaction is carried out at 37° C. with magnetic stirring. After 24 hours, the reaction is stopped by incubation at 95° C. for 10 minutes. The mixture is stored at −20° C. before purification.

A pre-purification step of the glucosylated hydroxy-lipids derived from HESUA is carried out by flash chromatography using a column containing 250 ml of PuroSorb PAD910 stationary phase, Purolite, USA. The glucosylation products are thus separated from residual free sugars by the following elution steps:
4 column volumes (CV) of ultrapure water until complete elution of the residual sugars
Elution of glycosylation products using 4 CV of a 35%/65% v/v H₂O/ethanol mixture.
Return to 100% ultrapure H₂O in 5 CV.

The glucosylation products are collected and concentrated by rotary evaporation under vacuum and then analyzed by chromatography. The chromatogram obtained in this pre-purification step is shown in FIG. 10. At least 6 main glucosylation products appear therein in reverse order of polarity. Product 1 corresponds to the one eluted closest to HESUA, lipid acceptor that has not completely reacted.

The predominant glucosylated forms of HESUA (glucosylation products 1 to 3) are isolated using a semi-preparative system consisting of an Agilent 1260 Infinite HPLC system coupled to a Dionex Ultimate 3000 automated fraction collector. Separation is provided by a Synergi™ Fusion-RP column (porosity of 80 Å, particle size of 4 μm, C18 grafting with polar termination, Phenomenex, USA). The elution is carried out isocratically with a 65%/35% v/v H₂O/acetonitrile mixture at a flow rate of 1 mL·min⁻¹.

### 4.1.2. Characterization of Glucosylation Products of HESUA

### A) High Resolution Mass Spectrum (LTQ Orbitrap) of HESUA Glucosylation Products

In order to determine the exact masses of the purified glucosylation products, the Thermo U3000 HPLC system was coupled to a high-resolution LTQ Orbitrap Velos spectrometer (Thermo Fisher Scientific, USA). Ionization is carried out in negative electrospray mode (HESI II-) with a mass delta of +/−5 ppm. The high-resolution mass spectra of glucosylation products 1 to 3 are obtained (FIG. 10).

The high-resolution LC-MS analysis of HESUA glucosylation product 1 with a retention time of 7.8 min leads to the identification of a major ion at m/z 423.20492 for [M-H]⁻ corresponding to a monoglucosylated form of HESUA.

A major ion obtained with glucosylation product 2 (elution time=6.5 minutes) at m/z 585.25716 for [M-H]⁻ corresponds to a di-glucosylated form of HESUA.

Surprisingly, glucosylation product 3 (peak 3 at 5.92 min) gives a principal ion at m/z 585.2571 for ([M-H]⁻). Compound 3 is therefore also a di-glucosylated form of HESUA but with a different glycosidic bond from that of product 2.

### b) NMR Analysis of HESUA Glucosylation Products 1 to 3

The HESUA glucosylation products 1 (monoglucosylated HESUA), 2 and 3 (di-glucosylated HESUA) are characterized by NMR. The ¹H, ¹³C, JMod, HSQC and HMBC spectra were recorded on Bruker Avance 500 MHz equipment at 298 K with a 5-mm BBI H-BB-D Z-GRD probe. The data were acquired and processed using TopSpin 3 software.

### NMR Characterization of HESUA Glucosylation Product 1

A ¹H spectrum and a ¹³C spectrum of the HESUA glucosylation product 1 are produced.

The ¹H and ¹³C spectra obtained with glucosylation product 1 are in agreement with a monoglucosylation of HESUA.

Peak 1 of the chromatograms shown in FIG. 10 corresponds to 11-[(2-O-α-D-glucopyranosyl-ethyl)-sulfanyl]-undecanoic acid.

### NMR Characterization of HESUA Glucosylation Product 2

A ¹H spectrum and a ¹³C spectrum of the HESUA glucosylation product 2 are produced. The presence of two anomeric protons at chemical shifts 4.84 and 5.13 ppm and two negative carbons at chemical shifts 100.54 ppm and 101.94 ppm (anomeric carbons) are consistent with a di-glucosylated form of the lipid acceptor.

An HSQC 2D spectrum of HESUA-product 2 allows the allocation of anomeric protons and carbons for each saccharide unit.

A 2D HMBC NMR spectrum of HESUA glycosylation product 2 shows an glycosidic bond of the α-1,3 type between the two glucosyl units.

Peak 2 of the chromatograms shown in FIG. 10 therefore corresponds to 11-[(2-O-α-D-glucopyranosyl-(1,3)-α-D-glucopyranosyl-ethyl) sulfanyl]-undecanoic acid.

### NMR Characterization of HESUA Glucosylation Product 3

A ¹H spectrum and a ¹³C spectrum of HESUA glucosylation product 3 are produced. The presence of two anomeric protons at chemical shifts 4.99 and 5.05 ppm and two negative carbons at chemical shifts 97.56 ppm and 98.28 ppm (anomeric carbons) are consistent with a second di-glucosylated form of the lipid acceptor.

An HSQC 2D spectrum of HESUA glucosylation product 3 allows the allocation of anomeric protons and carbons for each saccharide unit. A 2D HMBC spectrum of glucosylation product 3 shows a glycosidic bond of the α-1,2 type between the two glucosyl units.

Peak 3 of the chromatogram shown in FIG. 10 therefore corresponds to 11-[(2-O-α-D-glucopyranosyl-(1,2)-α-D-glucopyranosyl-ethyl) sulfanyl]-undecanoic acid.

### 4.2. Production and Structural Characterization of HUDA Glucosylation Products

### 4.2.1. Production of a Batch of HUDA Glycolipids at Gram Scale.

The production of glucosylation products is carried out by the enzyme BRS-B on 1 g of HUDA. The reaction conditions are as follows:
final sucrose concentration: 438.5 mM
final HUDA concentration: 10 mM (initially dissolved in DMSO at 100 mM)
sodium acetate buffer at 50 mM and at pH=5.75
ultrapure water qs 500 mL
activity of BRS-B in the reaction: 1 U·mL⁻¹

The reaction is carried out at 30° C. with magnetic stirring. After 24 hours, the reaction is stopped by incubation at 95° C. for 10 minutes. The mixture is stored at −20° C. before purification.

### 4.2.2. Purification of HUDA Glycolipids Produced by the Enzyme BRS-B.

A pre-purification step of the glycolipids derived from HUDA is carried out by flash chromatography using a REVELERIS® X2 Flash Chromatography System (GRACE, USA) equipped with a column containing 80 g of silica-C18 stationary phase. Glucosylation products are separated from residual free sugars under the following conditions:
flow rate of 60 mL·min⁻¹
3 column volumes (CV) at 100% H₂O
gradient going from 100% ultrapure H₂O to 100% acetonitrile in 2 CV
1 CV at 100% acetonitrile
return to 100% ultrapure H₂O in 0.5 CV
equilibration at 100% ultrapure H₂O during 1.5 CV

The peak corresponding to the glucosylation products is collected during the acetonitrile gradient. The chromatogram obtained during this pre-purification step by flash chromatography is shown in FIG. 11.

Negative electrospray LC-MS analysis of the glucosylation product of HUDA 1 (retention time of 15.34 min) gives two major ions at m/z 363.2 for [M-H]⁻ corresponding to the monoglucosylated form and m/z 725.3 for [2M-3H]⁻ corresponding to a dimer.

Negative electrospray LC-MS analysis of the glucosylation product of HUDA 2 (retention time of 14.12 min) gives a major ion at m/z 525.3 for [M-H]⁻ corresponding to the di-glucosylated form.

The different glucosylated forms of HUDA are then purified on a semi-preparative system consisting of an Agilent 1260 Infinite HPLC system coupled to a Dionex ultimate 3000 automated fraction collector. Separation is provided by a Synergi™ Fusion-RP column (porosity of 80 Å, particle size of 4 μm, C18 grafting with polar termination, Phenomenex, USA). The elution is carried out isocratically with a 75%/25% v/v H₂O/acetonitrile mixture at a flow rate of 1 mL·min⁻¹.

### 4.2.3. NMR Analysis of Purified HUDA Glycolipids.

The characterization of the glucosylation products of HUDA 1 (monoglucosylated HUDA) and 2 (di-glucosylated HUDA) is carried out by NMR. The 1H, 13C, JMod, HSQC and HMBC spectra were recorded on Bruker Avance 500 MHz equipment at 298 K with a 5-mm BBI H-BB-D Z-GRD probe. The data were acquired and processed using TopSpin 3 software.

### NMR Characterization of HUDA Glucosylation Product 1

A ¹H spectrum and a ¹³C spectrum of HUDA glucosylation product 1 are produced.

The ¹H and ¹³C spectra obtained with HUDA glucosylation product 1 are in agreement with a monoglucosylation of HUDA.

Peak 1 of the chromatogram shown in FIG. 11 corresponds to 11-O-(α-D-glucopyranosyl)-undecanoic acid.

### 4.3. Production and Structural Characterization of Glucosylation Products of EAA by BRS-B Δ1

### 4.3.1. Production of Batches of Glycosylated Erythro-Aleuritic Acid Derivatives at Gram Scale.

The production of glucosylation products of EAA is carried out by the enzyme BRS-B Δ1 on 1.5 g of EAA. The reaction conditions are as follows:
Final sucrose concentration: 584.7 mM
Final EAA concentration: 10 mM (initially dissolved in DMSO at 100 mM)
Sodium acetate buffer at 50 mM and at pH=5.75
Ultrapure water qs 500 mL
Activity of BRS-B in the reaction: 1 U·mL⁻¹

The reaction is carried out at 30° C. with magnetic stirring. After 24 hours, the reaction is stopped by incubation at 95° C. for 10 minutes. The mixture is stored at −20° C. before purification.

4.3.2. Purification of Glycolipids from EAA Produced by the Enzyme BRS-B Δ1.

A pre-purification step of the glycolipids derived from EAA is carried out by flash chromatography using a REVELERIS® X2 Flash Chromatography System (GRACE, USA) equipped with a column containing 80 g of silica-C18 stationary phase. Glucosylation products are separated from residual free sugars by the following elution steps at a flow rate of 60 mL·min⁻¹:
3 column volumes (CV) of 100% ultrapure H₂O.
Gradient going from 100% ultrapure H₂O to 100% acetonitrile in 2 CV
1 CV at 100% acetonitrile.
Return to 100% ultrapure H₂O in 0.5 CV
Re-equilibration at 100% ultrapure H₂O during 1.5 VC

The glucosylation products are collected during the acetonitrile gradient.

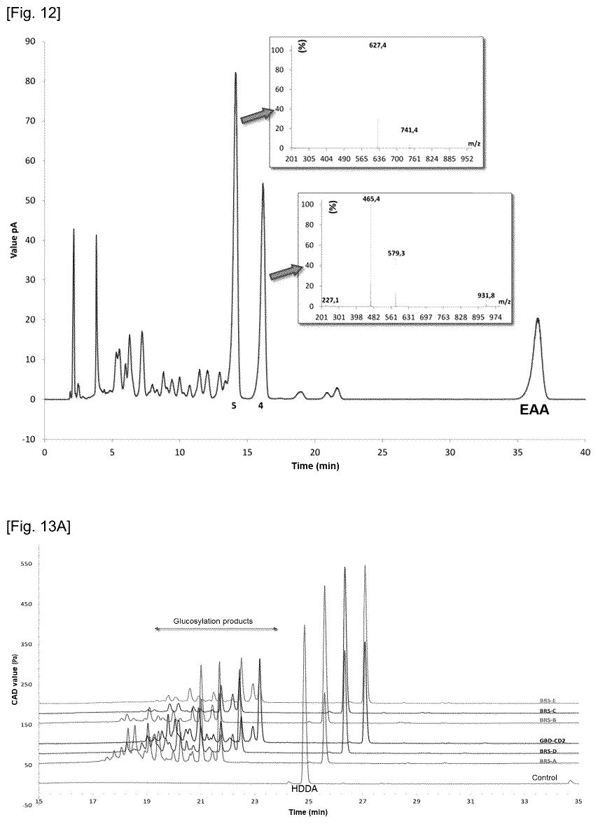

FIG. 12 illustrates the chromatogram obtained during this flash chromatography pre-purification step.

LC-MS analysis in negative electrospray mode of EAA glucosylation product 4 whose retention time is 16.16 min leads to two major ions at m/z 465.4 [M-H]⁻ and m/z 579.3 [M+TFA−H]⁻. These ions confirm the production of a monoglucosylated form of EAA.

The LC-MS analysis in negative electrospray mode of the glucosylation product EAA 5 whose retention time is 14.12 min leads to two major ions at m/z 627.4 [M-H]⁻ and m/z 741.4 [M+TFA-H]⁻). These two ions at +162 amu with respect to peak 4 (m/z 465.4) confirm the obtaining of a di-glucosylated form of EAA.

These two main glucosylated forms of erythro-aleuritic acid are then purified on a semi-preparative system consisting of an Agilent 1260 Infinite HPLC system coupled to a Dionex ultimate 3000 automated fraction collector. Separation is provided by a Synergi™ Fusion-RP column (porosity of 80 Å, particle size of 4 μm, C18 grafting with polar termination, Phenomenex, USA). The elution is carried out isocratically with a 78%/22% v/v H₂O/acetonitrile mixture at a flow rate of 1 mL·min⁻¹.

### 4.3.3. NMR Analysis of Purified EAA Glucosylation Products.

The characterization of EAA glucosylation products 4 (monoglucosylated EAA) and 5 (di-glucosylated EAA) is carried out by NMR. The 1H, ¹³C, HSQC and HMBC spectra were recorded on Bruker Avance 500 MHz equipment at 298 K with a 5-mm BBI H-BB-D Z-GRD probe. The data were acquired and processed using TopSpin 3 software.

### Characterization of EAA Glycosylation Product 4 (Monoglucosylated EAA)

A ¹H spectrum and a ¹³C spectrum of EAA glucosylation product 1 are produced. The ¹H and ¹³C spectra obtained with EAA glucosylation product 4 are in agreement with a monoglucosylation of EAA.

The ¹H spectrum reveals an anomeric proton at a chemical shift of 4.77 ppm. The ¹³C spectrum shows the presence of negative carbon peaks C9 and C10 superimposed at a chemical shift of 75.38 ppm, which proves the absence of glucosylation on the two secondary hydroxyls. Glucosylation is therefore at unshielded positive carbon 16 at 69.21 ppm.

Peak 4 of the chromatogram shown in FIG. 12 corresponds to 16-O-(α-D-glucopyranosyl)-9,10-dihydroxy-hexadecanoic acid.

### Characterization of EAA Glucosylation Product 5 (Di-Glucosylated EAA)

A ¹H spectrum and a ¹³C spectrum of EAA glucosylation product 5 are produced.

The ¹H and ¹³C spectra obtained with EAA glucosylation product 4 are in agreement with a di-glucosylation of EAA.

The ¹H spectrum reveals two anomeric protons at a chemical shift of 4.79 ppm and 5.14 ppm and two anomeric carbons at 100.34 ppm and 101.89 ppm. Like the monoglucosylated form, the negative carbon peaks C9 and C10 are still present at 75.41 ppm, demonstrating di-glucosylation at the C16 position.

The 2D HMBC spectrum of EAA shows a glycosidic bond of the α-1,3 type between the two glucosyl units.

Peak 5 of the chromatogram shown in FIG. 12 corresponds to a di-glucosylated EAA of the 16-O-(α-D-glucopyranosyl)-(1,3)-α-D-glucopyranosyl)-9,10-dihydroxyhexadecanoic acid type.

### Example 5: Glucosylation of Omega-Hydroxy Fatty Acids Having a Hydrocarbon Chain with a Carbon Number Greater than 11

The branching enzymes BRS-A, BRS-B Δ1, BRS-C, BRS-D Δ1, BRS-E Δ1 and GBD-CD2 ΔN123 were tested for glucosylating 12-hydroxydodecanoic acids (HDDA), 15-hydroxypentaecanoic acids (HPDA) and 16-hydroxyhexahecanoic acids (HHDA) comprising 12, 15 and 16 carbon atoms, respectively.

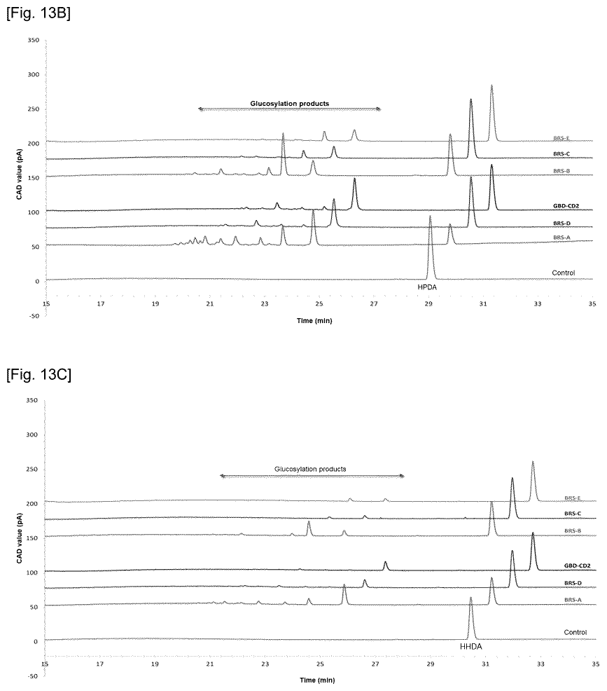

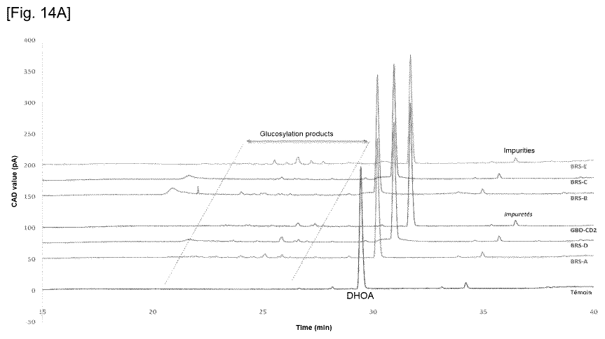

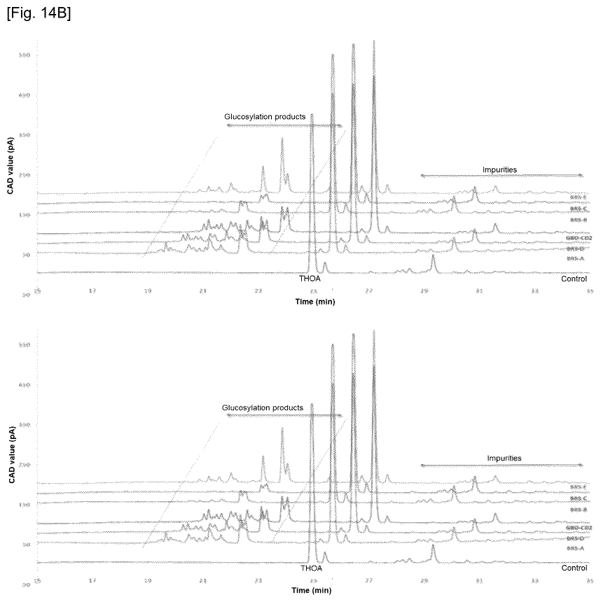

FIG. 13 shows the glucosylation products obtained at 37° C. under 800 rpm stirring under the following synthesis conditions:
Initial concentration of sucrose: 877 mM
Initial concentration of omega-hydroxy acid: [HDDA]=10 mM; [HPDA]=1 mM and [HHDA]=0.5 mM,
10% v/v of DMSO
50 mM sodium acetate buffer at pH=5.75
Enzymatic activity: 1 U·mL⁻¹

For HPLC analysis, the reaction media are diluted to half in absolute ethanol. Separation is carried out by a Synergi™ Fusion-RP column (porosity of 80 Å, particle size of 4 μm, C18 grafting with polar termination, Phenomenex, USA). The elution is carried out at 30° C. on a Thermo U3000 HPLC system coupled to a Corona CAD Veo (charged aerosol detector) (Thermo Fisher Scientific, USA). The nebulization temperature is set at 50° C. and the filter is maintained at 3.2 seconds.

The mobile phase is composed of a mixture of ultrapure water (solvent A) and HPLC grade acetonitrile (solvent B) containing 0.05% (v/v) formic acid. Elution is carried out at a flow rate of 1 mL·min⁻¹ according to the following gradient:
Between 0 min and 5 min, a first phase of elution at 0% (v/v) of channel B.
A gradient phase going from 0% of channel B to 100% of channel B in 35 minutes.

Analysis of the two chromatograms presented in FIG. 14 demonstrates the possibility of glucosylating long chain fatty acids bearing primary hydroxyl in position @ (up to at least C₁₆).

Indeed, the comparison of the initial (control) and final reaction times shows the appearance of numerous glucosylation products between the elution times 17 minutes and 22 minutes for HDDA, between 19 minutes and 25 minutes for HPDA and between 21 minutes and 27 minutes for HPDA.

Glucosylation efficiency is dependent on the chain size of the ω-hydroxy acceptors considered. Glucosylation of acceptors of longer chain length is obtained solely by reducing the working concentrations (from 10 mM for HDDA to 0.5 mM for HHDA, for example).

### Glucosylation of 12-Hydroxydodecanoic Acid (HDDA)

The branching enzymes BRS-A, BRS-D Δ1, GBD-CD2 ΔN123 (specific for the α-1,2 bond) and the enzyme BRS-E (specific for the α-1,3 bond) seem to be the most efficient enzymes in this case.

Indeed, the conversion rates are for HDDA, 60%, 40% and 42% for the enzymes BRS-A, BRS-D Δ1 and GBD-CD2 ΔN123, respectively, and only 18%, 11% and 17% for the enzymes BRS-B Δ1, BRS-C and BRS-E Δ1, respectively.

The chromatograms obtained show multi-glucosylation profiles with numerous peaks, two of which are predominantly eluted at 21 and 22 minutes.

### Glucosylation of 15-Hydroxypentadecanoic Acid (HPDA) and 16-Hydroxyhexadecanoic Acid (HHDA)

Glucosylation of longer chain acceptors is more efficient with the two branching enzymes BRS-A (α-1,2 specific) and BRS-B (α-1,3 specific). Indeed, the conversion rates obtained with these acceptors are:
for HPDA, 70% for BRS-A and 33% for BRS-C. The conversion efficiencies of HPDA are only 5.0%, 22%, 13% and 29% for BRS-C, BRS-D Δ1, BRS-E Δ1 and GBD-CD2 ΔN123, respectively.

For HHDA, 38% for BRS-A and 21% for BRS-C. The conversion efficiencies for HHDA are only 5%, 12%, 6% and 12% for BRS-C, BRS-D Δ1, BRS-E Δ1 and GBD-CD2 ΔN123, respectively.

### Example 6: Glucosylation of 9,10-Dihydroxyoctadecanoic Acids and 9,10,12-Trihydroxyoctadecanoic Acids by the Enzymes BRS-A, BRS-B Δ1, BRS-C, BRS-D Δ1, BRS-E Δ1 and GBD-CD2 ΔN123

The enzymes BRS-A, BRS-B Δ1, BRS-C, BRS-D Δ1, BRS-E Δ1 and GBD-CD2 ΔN123 (branching α-transglucosylase) were tested to glucosylate 9,10-dihydroxyoctadecanoic acids (DHOA) and 9,10,12-trihydroxyoctadecanoic acids (THOA).

FIG. 14 shows the glucosylation products obtained at 37° C. under 800 rpm stirring under the following synthesis conditions:
Initial concentration of sucrose: 877 mM
Initial concentration of poly-hydroxy fatty acid (DHOA and THOA): 5 mM,
10% v/v of DMSO
50 mM sodium acetate buffer at pH=5.75
Enzymatic activity: 1 U·mL⁻¹

For HPLC analysis, the reaction media are diluted to half in absolute ethanol. Separation is carried out by a Synergi™ Fusion-RP column (porosity of 80 Å, particle size of 4 μm, C18 grafting with polar termination, Phenomenex, USA). The elution is carried out at 30° C. on a Thermo U3000 HPLC system coupled to a Corona CAD Véo (charged aerosol detector) (Thermo Fisher Scientific, USA). The nebulization temperature is set at 50° C. and the filter is maintained at 3.2 seconds.

The mobile phase is composed of a mixture of ultrapure water (solvent A) and HPLC grade acetonitrile (solvent B) containing 0.05% (v/v) formic acid. Elution is carried out at a flow rate of 1 mL·min⁻¹ according to the following gradient:
Between 0 min and 5 min, a first phase of elution at 0% (v/v) of channel B.
A gradient phase going from 0% of channel B to 100% of channel B in 30 minutes.

The analysis of the two chromatograms presented in FIG. 14A and FIG. 14B proves the possibility of glucosylating fatty acids bearing secondary hydroxyl present only within the carbon chain.

Indeed, the comparison of the initial (control) and final reaction times shows the appearance of numerous glucosylation products between the elution times 21 minutes and 26 minutes for DHOA and 19 minutes and 23 minutes for THOA. The formation of these glucosylation products appears much more efficient with trihydroxy fatty acid (THOA) than with the dihydroxy form DHOA, the intensity of the observed peaks being much greater in the first case (THOA).

Similarly, conversion rates vary between 2% and 30% for THOA and between 1% and 11% for DHOA depending on the branching enzymes considered.

The branching enzymes BRS-A, BRS-D Δ1, GBD-CD2 ΔN123 (specific for the α-1,2 bond) and BRS-E (specific for the α-1,3 bond) appear to be the most efficient enzymes in both cases.

### Example 7: Glucosylation of Hydroxy Fatty Acids by Mutants of the Enzyme GBD CD2 ΔN123

Mutants of the enzyme GBD CD2 ΔN123 were tested to glucosylate the following hydroxy fatty acids:
11-[2-[hydroxy-alkyl)-sulfanyl]-undecanoic acid (HESUA),
11-Hydroxyundecanoic acid (HUDA)
12-hydroxydodecanoic acid (HDDA),
15-hydroxypentanoic acid (HPDA),
erythro-aleuritic acid (EAA),
9,10,12-trihydroxy octadecanoic acid (THOA).

The results show that these mutants perform very well insofar as they exhibit very higher conversion rates. Conversion rates of the following hydroxy fatty acids are obtained:

| SEQ ID | | Glucosylated compound | | | | | |
|---|---|---|---|---|---|---|---|
| No. | Name | HESUA | HUDA | HDDA | HPDA | EAA | THOA |
| 1 | GBD-CD2 ΔN123 WT | 70% | 49% | 42% | 16% | 80% | 4% |
| 7 | GBD-CD2 ΔN123 | 98% | 86% | 79% | 36% | 93% | 68% |
| | W2135L-F2136L | | | | | | |
| 8 | GBD-CD2 ΔN123 | >99% | 96% | 96% | 33% | 94% | 32% |
| | W2135L | | | | | | |
| 9 | GBD-CD2 ΔN123 | >99% | 93% | 94% | 31% | 79% | 15% |
| | W2135I-F2136Y | | | | | | |
| 10 | GBD-CD2 ΔN123 | 98% | 86% | 84% | 33% | 84% | 77% |
| | W2135I-F2136C | | | | | | |
| 11 | GBD-CD2 ΔN123 | 99% | 98% | 99% | 42% | 90% | 19% |
| | W2135V | | | | | | |

Conversion rate obtained with GBD CD2 ΔN123 mutants.

GBD-CD2 ΔN123 mutants also make it possible to obtain glucosylation products having larger saccharide motifs.

### Example 8: Study of the Emulsifying Properties of Glucosylation Products of HESUA by BRS-A and HUDA by BRS-B Δ1

### 8.1. Preparing the Emulsions

The emulsifying properties of the HESUA glucosylation products by the enzyme BRS-A (hereinafter referred to as Glc-HESUA) and of HUDA by BRS-B Δ1 (hereinafter Glc-HUDA) were evaluated.

### Aqueous Phase

An aqueous solution comprising 2% by mass of Glc-HESUA is prepared.

The pH of the aqueous solution is adjusted using a 1 M NaOH solution (the pH values studied are 4, 5, 6 and 10.5).

These different pH values make it possible to scan the different ionization states of the carboxyl function of the compounds which are below, near and above the pKa (mean pKa of Glc-HESUA=4.88; mean pKa of Glc-HUDA=5, determined by acid-base assay).

### Oil Phase

Dodecane is used as a model oil.

### Emulsification

The oil is slowly added to the aqueous solution while homogenizing with an Ultra Turrax T25 (Janke & Kunkel, IKA) set at 3000 rpm.

The final percentage of oil is 20% by mass.

The preparation is then stirred for 10 minutes, increasing the speed of the Ultra Turrax to 9,000 rpm.

The kinetic evolution of the emulsions thus formed is monitored over time by laser particle size analysis (static light scattering) (Mastersizer 2000, Malvern Instruments), light microscopy and macroscopic observation.

The particle size distribution of the emulsions is characterized in terms of the volume mean diameter of the drops, D (4.3), and of the polydispersity, P, defined above ([Chem 1] and [Chem 2]).

8.2. Emulsions Obtained with Glc-HESUA
8.2.1. Characterization of Emulsions Obtained with Glc-HESUA

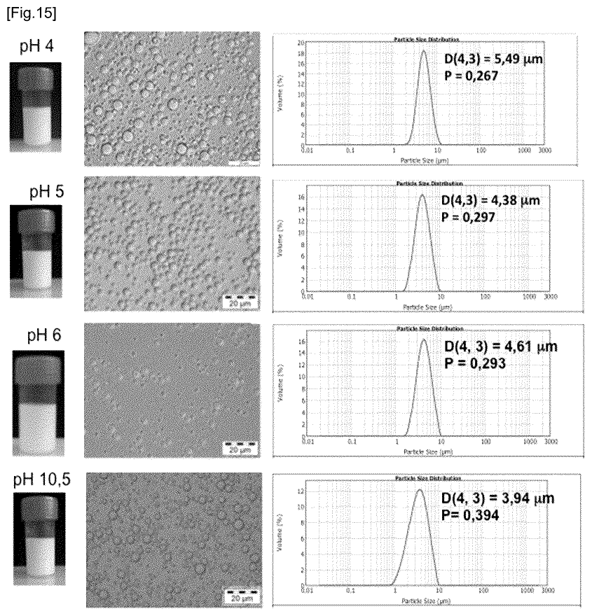

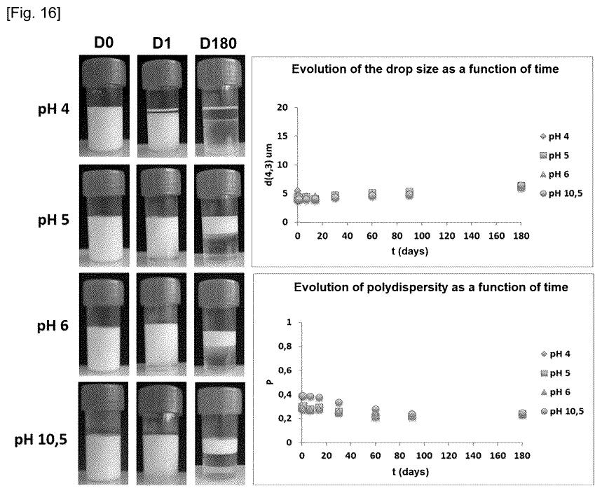

FIG. 15 shows the characteristics of the emulsions on the day of their preparation (day 0) at the various pH values studied.

The emulsions formed have a milky white appearance. The initial particle sizes are generally quite close regardless of the pH: The mean diameter is around 4-5 μm and the polydispersity is between 0.27 and 0.49. The emulsion at pH=10.5 is more polydisperse than the others with a widening toward small diameters.

The emulsion at pH=4 destabilizes by coalescence (fusion between drops) in the hours following its manufacture and is destroyed after 3 days (macroscopic separation of the oil and the aqueous phase) (FIG. 16). Emulsions at pH=5, 6 and 10.5 are kinetically stable over time for at least 6 months. The size of the drops remains virtually unchanged as shown by the monitoring of the diameter of the drops D (4.3) as a function of time (FIG. 16).

A creaming phenomenon can also be noted (FIG. 16), which consists of the concentration of the drops of oil on the surface of the emulsion under the effect of gravity, the drops having a density lower than that of the aqueous phase.

The glucosylation product Glc-HESUA therefore makes it possible to stabilize emulsions whose aqueous phase has a pH greater than the pKa. The instability observed at low pH reflects the existence of a strong electrostatic component in the stabilization of emulsions. The electrostatic repulsion that prevents coalescence (recombination) of the drops results from charged surfactant molecules adsorbed at the interfaces.

8.2.2 Study of the Destabilization of an Emulsion Obtained with Glc-HESUA by pH Modulation

Due to the presence of an acid function on Glc-HESUA and the pH dependence of the stability of the emulsions described above (coalescence observed rapidly at pH=4), it is possible to envisage obtaining pH-stimulable emulsions, i.e., stable in a pH range and destabilizing at a given pH, “on demand”.

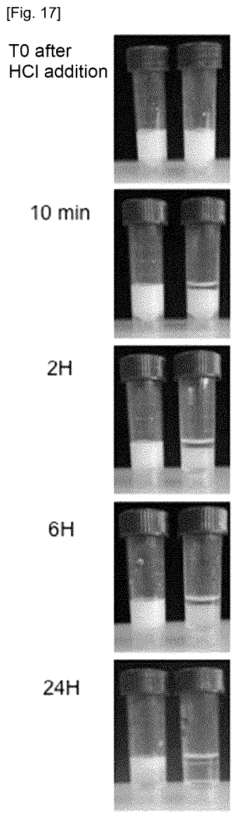

Tests were carried out for this purpose (FIG. 17).

The initial emulsion is prepared at pH=6, pH where the emulsion is stable over time as shown above. An HCl solution (0.1 M) is then added to bring the pH down to 2. The emulsion is completely destroyed after 24 hours.

The emulsions can also be stimulated by addition of salt. Indeed, an emulsion at pH 10.5 prepared with 0.1 M NaCl destabilizes after five days while it is still stable after 6 months in the absence of salt. This supports the hypothesis that stabilization is of electrostatic origin insofar as it is known that the addition of salt weakens electrostatic repulsion.

8.3. Properties of Emulsions Obtained with Glc-HUDA
8.3.1. Characterization of Emulsions Obtained with Glc-HUDA

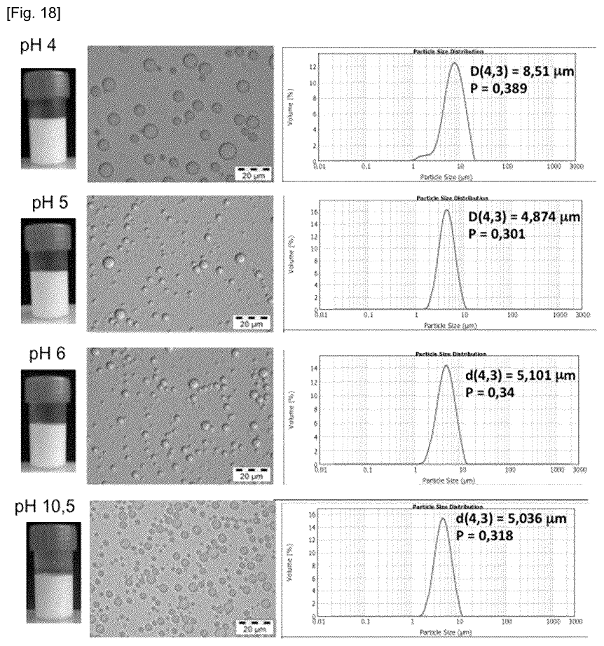

FIG. 18 shows the characteristics of the emulsions on the day of their preparation (day 0) at the various pH values studied.

The emulsions formed have a milky white appearance. The initial particle sizes are generally fairly close for pH values above 5: The mean diameter is around 4-5 μm and the polydispersity is between 0.30 and 0.34. It will be noted that at pH=4 the mean diameter of the drops is larger (8.51 μm), with a polydispersity of 0.39.

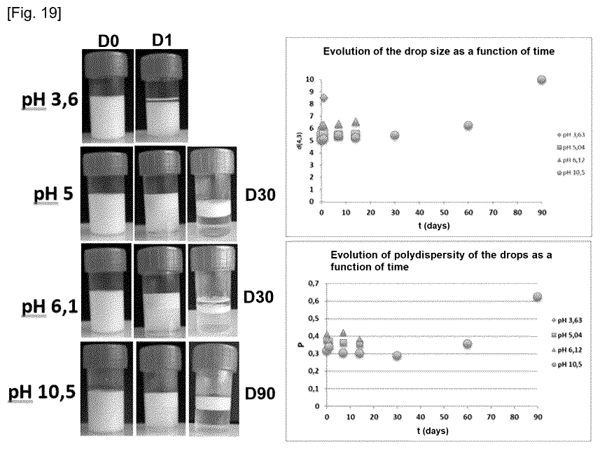

The emulsion at pH=3.6 is destroyed after 1 day (FIG. 19). The emulsions at pH=5, 6.1 and 10.5 are kinetically stable over time for at least 14 days.

The size of the drops remains virtually unchanged up to 14 days as shown by the monitoring of the diameter of the drops D (4.3) as a function of time (FIG. 19). A thin layer of oil is observed on the emulsions at pH 5 and pH 6.1 between 14 and 30 days, reflecting destabilization of the emulsion by coalescence. The particle size measurements are interrupted as soon as this layer becomes visually perceptible. For pH 10.5, a gradual increase in the size of the drops without the macroscopic aspect being impacted at this stage is observed. Follow-up at 6 months shows the appearance of a thin layer of oil. For all the emulsions, a creaming phenomenon is noted, which consists of the concentration of the drops of oil on the surface of the emulsion under the effect of gravity, the drops having a density lower than that of the aqueous phase. This phenomenon can be limited by adding a thickener to the aqueous phase.

Glc-HUDA therefore acts as a surfactant stabilizing emulsions at pH values above pKa and more particularly at strongly basic pH (greater stability at pH 10.5 than at pH 6). The instability observed at low pH reflects the existence of a strong electrostatic component in the stabilization of emulsions. The electrostatic repulsion that prevents coalescence of the drops results from charged surfactant molecules adsorbed at the interfaces.

8.3.2. Study of the Destabilization of an Emulsion Obtained with Glc-HUDA by pH Modulation

Glc-HUDA makes it possible to manufacture pH-stimulable emulsions, i.e., are stable in a pH range and that destabilize at a given pH, “on demand”.

Preliminary tests were carried out for this purpose (FIG. 20). The initial emulsion is prepared at pH=6, pH where the emulsion is stable over time as shown above. An HCl solution (0.1 M) is then added to bring the pH down to 2. The emulsion is completely destabilized after two hours.

8.4. Self-Motility Properties of Emulsions Prepared with Glc-HESUA and Glc-HUDA

A self-motility phenomenon was observed with an emulsion prepared with Glc-HESUA and Glc-HUDA.

Indeed, when a drop of emulsion (approximately 20 μL) is deposited on a solid substrate (glass slide, plastic support, support with modified hydrophobicity), it is observed that it is subjected to very intense convective phenomena. The drop not only moves but spreads and contracts spasmodically and periodically on its support.

A phenomenon consisting of the sudden appearance/disappearance of transparent zones in the vicinity of the surface of the emulsion was also observed with the emulsions prepared with Glc-HESUA and Glc-HUDA.

Indeed, in certain cases, zones that are transparent (non-milky), and therefore depleted of drops, form spontaneously in the vicinity of the surface of the drop and disappear immediately (phenomenon similar to boiling without bubbles forming, however). Again, the phenomenon seems to occur with a certain periodicity of around a few seconds to a few minutes depending on the composition of the systems.

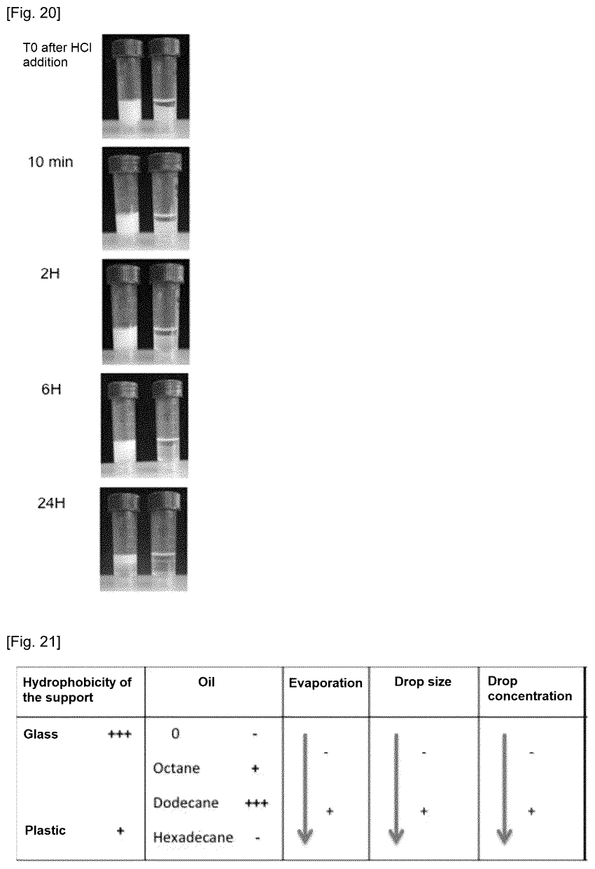

These phenomena depend on many factors: evaporation, nature of the support, size of the dispersed phase drops and concentration, nature of the oil as shown in FIG. 21 which summarizes the influence of these parameters on the intensity of these phenomena.

These phenomena are probably the manifestation of the Marangoni effect, under various forms (R. T. van Gaalen, C. Diddens, H. M. A. Wijshoff, J. G. M. Kuerten (2021). Marangoni circulation in evaporating droplets in the presence of soluble surfactants, Journal of Colloid and Interface Science, Volume 584, Pages 622-633). This results in the convection of matter on a large scale, following the appearance of an interfacial tension gradient.

Such emulsions have the advantage of being self-spreadable and self-stirring, which makes it possible to foresee different applications in the field of surface cleaning.

### Example 9: Study of the Preparation of Dry Emulsions Using Glycolipids According to the Invention

According to another embodiment, the composition is in the form of a dry powder, said dry powder being advantageously redispersible in water. The dry redispersible form has numerous advantages, such as ease of transport and prolonged shelf life due to the absence of bacteriological development. Surfactant-stabilized emulsions generally destabilize during drying (oil exudation). Advantageously, the emulsions based on glycolipids synthesized in the present invention can be dried without destabilizing and are redispersible in water.

### 9.1. Materials and Methods

### 9.1.1. Preparation of a Primary Emulsion

### Aqueous Phase

An aqueous solution comprising 2% by mass of Glc-HESUA and 10% lactose is prepared. Lactose is added to the aqueous phase as cryoprotective agent.

The pH of the aqueous solution is adjusted to pH=6 using an NaOH solution.

### Oil Phase

Dodecane is used as a model oil.

### Emulsification

The oil is slowly added to the aqueous solution while homogenizing with an Ultra Turrax T25 (Janke & Kunkel, IKA) set at 3000 rpm.

The percentage of oil is 20% by mass.

The preparation is then stirred for 10 minutes, increasing the speed of the Ultra Turrax to 9,500 rpm.

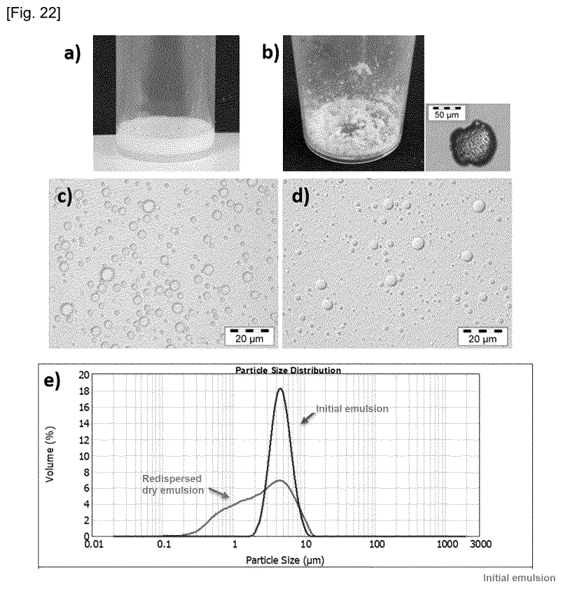

A primary emulsion is obtained. Its mean diameter is 4.94 μm (P=0.27) (FIG. 22).

### 9.2.2. Preparation of the Dry Emulsion

A dry emulsion is obtained by lyophilizing the primary emulsion previously frozen at −80° C.

### 9.2.3. Redispersion of the Dry Emulsion

The dry emulsion is redispersed by adding pure water in an amount equivalent to that evaporated during drying.

### 9.3. Results

The results show that a primary emulsion prepared with Glc-HESUA can be put into the form of a dry emulsion and redispersed with gentle stirring. No oil exudation is observed after drying and the mean diameter of the redispersed emulsion is less than 10 μm (FIG. 22).

### Example 10: Antimicrobial Properties of Glucosylation Products

### 10.1. Materials and Methods

E. coli K12 strains are cultured in the presence of 0% w/v, 0.5% w/v, 1% w/v, 2% w/v and 2.5% w/v aqueous glucosylation products of HUDA or EAA.

The absorbance of cultures in the presence of glucosylation products of HUDA or EAA is monitored at an optical density of 600 nm.

### 10.2. Results

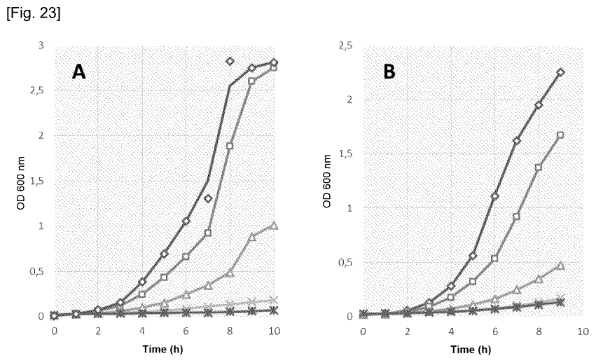

The results (FIG. 23) show that the glucosylation products of HUDA and EAA are bacteriostatic for contents of 1% w/V.

1. A method for preparing at least one glycolipid corresponding to formula (I):
[Glc]n-xOy-R (I)
wherein
[Glc]ₙ represents a linear or branched saccharide motif comprising n glucosyl units,
with n comprised between 1 and 7,
with the condition that, when the saccharide motif comprises several glucosyl units, these units are linked together by α-type glycosidic bonds;
R represents a fatty acid radical comprising between 4 and 24 carbon atoms, the carbon chain of which is linear or branched, saturated or unsaturated, optionally interrupted by one or more sulfur atoms, and optionally comprising one or more substituent(s) chosen from a hydroxyl group, carbonyl group, methoxyl group, amine group, nitrosyl group and thiol group,
-xOy- symbolizes the attachment of the fatty acid radical to the saccharide motif by an ether bond linking a Cx carbon atom of a glucosyl residue of the saccharide motif, previously bearing a hydroxyl group, to a Cy carbon atom of the fatty acid radical, previously bearing a hydroxyl group, with Cx being the position of the atom of the glucosyl residue on which the bond is effected, Cx representing the C₁ carbon atom of the glucosyl residue, Cy being a carbon atom positioned along the carbon chain of the fatty acid radical or at the omega end thereof;
said method comprising a step of glucosylation of a hydroxy fatty acid of formula (II):
R-yOH (II)
wherein
R is a fatty acid radical as defined in formula (I),
-yOH represents a hydroxyl group attached to a Cy carbon atom as defined above;
said step comprising contacting the hydroxy fatty acid of formula (II) with at least one-transglucosylase of the GH70 family in the presence of sucrose or a sucrose analog.

2. The method of claim 1, wherein the GH70 family α-transglucosylase is a branching sucrase of the GH70 family, a glucansucrase of the GH70 family, or a mixture thereof.

3. The method of claim 1, wherein the branching sucrase of the GH70 family has as its amino acid sequence a sequence chosen from the group comprising GBD-CD2 ΔN123 (SEQ ID NO:1), BRS-A (SEQ ID NO:2), BRS-B Δ1 (SEQ ID NO:3), BRS-C(SEQ ID NO: 4), BRS-D Δ1 (SEQ ID NO:5), BRS-E Δ1 (SEQ ID NO:6), or an amino acid sequence having at least 70%, 75%, 80%, 90%, 91%, 92%, 93%, 94%, 95%, 96%, 97%, 98%, 99% of sequence homology to at least one of SEQ ID NO: 1 to SEQ ID NO:6.

4. The method of claim 3, wherein the branching sucrase of the GH70 family has as its amino acid sequence a sequence chosen from the group comprising GBD-CD2 ΔN123 W2135L F2136L (SEQ ID NO:7), GBD-CD2 ΔN123 W2135L (SEQ ID NO:8), GBD-CD2 ΔN123 W21351 F2136Y (SEQ ID NO:9), GBD-CD2 ΔN123 W21351 F2136C (SEQ ID NO:10), GBD-CD2 ΔN123 W2135V (SEQ ID NO:11), or an amino acid sequence having at least 70%, 75%, 80%, 90%, 91%, 92%, 93%, 94%, 95%, 96%, 97%, 98%, 99% sequence homology with at least one of SEQ ID NO:7 to SEQ ID NO: 11.

5. The method of claim 1, wherein step i) is carried out with a molar ratio of sucrose or sucrose analog to hydroxy fatty acid of formula (II) comprised between 1 and 100.

6. Glycolipid corresponding to formula (I):
[Glc]n-xOy-R (I)
wherein
[Glc]n represents a linear or branched saccharide motif comprising n glucosyl units,
with n comprised between 1 and 7,
with the condition that, when the saccharide motif comprises several glucosyl units, these units are linked together by α-type glycosidic bonds;
R represents a fatty acid radical comprising between 4 and 24 carbon atoms, the carbon chain of which is linear or branched, saturated or unsaturated, optionally interrupted by one or more sulfur atoms, and optionally comprising one or more substituents chosen from a hydroxyl group, carbonyl group, methoxyl group, amine group, nitrosyl group and thiol group,
-xOy- symbolizes the attachment of the fatty acid radical to the saccharide motif by an ether bond linking a Cx carbon atom of a glucosyl residue of the saccharide motif, previously bearing a hydroxyl group, to a Cy carbon atom of the fatty acid radical, previously bearing a hydroxyl group, with Cx being the position of the atom of the glucosyl residue on which the bond is effected, Cx representing the C1 carbon atom of the glucosyl residue, Cy being a carbon atom positioned along the carbon chain of the fatty acid radical or at the omega end thereof.

7. The glycolipid of claim 6 wherein the Cy carbon atom is a carbon atom positioned at the omega end of the fatty acid radical.

8. The glycolipid of claim 6, wherein the glycolipid is of formula (Ia):
[Glc]ₙ-xOy-(CH₂)_{b}—S—(CH₂)ₐ—COOH
wherein
a is comprised between 9 and 14,
b is greater than or equal to 2, and
the sum of a and b is less than 23.

9. The glycolipid of claim 6, wherein the glycolipid has the formula (Ib):
[Glc]ₙ-xOy-(CH₂)ₐ—COOH (Ib)
wherein a is comprised between 3 and 23.

10. The glycolipid of claim 6, wherein the fatty acid radical is substituted by one or two hydroxyl groups, and preferably wherein:
the Cy carbon atom is a carbon atom positioned at the omega end, and the carbon chain of the fatty acid radical is substituted by one or two hydroxyl groups positioned along the carbon chain, or
the Cy carbon atom is a carbon atom positioned along the carbon chain, and the carbon chain of the fatty acid radical is substituted by two hydroxyl groups, with one hydroxyl group positioned along the carbon chain and one hydroxyl group positioned in the omega position of the carbon chain, or with two hydroxyl groups positioned along the carbon chain.

11. A method for destroying microbes or slowing their growth comprising using a glycolipid of formula (I) as defined in claim 6, as an antimicrobial agent.

12. A method for modifying the surface tension between two surfaces or for facilitating the formation of an emulsion or for improving its stability by reducing its rate of aggregation and/or coalescence, comprising using a glycolipid of formula (I) as defined in claim 6, as a surfactant.

13. An oil-in-water emulsion comprising an oil phase dispersed in an aqueous phase and at least one glycolipid of formula (I) such as defined in claim 6, wherein the emulsion is kinetically stable when the pH of the aqueous phase is greater than a threshold potential of hydrogen pHₛ.

14. The emulsion of claim 13 in the form of a dry emulsion.

15. A method for cleaning surfaces, comprising using an emulsion of claim 14 for cleaning the surfaces.

16. The method of claim 5, wherein step i) is carried out with a molar ratio of sucrose or sucrose analog to hydroxy fatty acid of formula (II) of 10 to 100.

17. The glycolipid of claim 9, wherein a is 10 to 15.

18. The oil-in-water emulsion of claim 13, wherein pHₛ is 2 to 5.
